(19) United States
(12) Patent Application Publication
(10) Pub. No.: US 2024/0085407 A1
(43) Pub. Date: Mar. 14, 2024

(54) BIOMATERIAL SENSOR SYSTEMS

(71) Applicant: The Regents of the University of Michigan, Ann Arbor, MI (US)

(72) Inventors: Aaron Morris, Ann Arbor, MI (US); Lonnie D. Shea, Ann Arbor, MI (US); Ariella Shikanov, Ann Arbor, MI (US)

(21) Appl. No.: 17/768,527

(22) PCT Filed: Oct. 19, 2020
(86) PCT No.: PCT/US2020/056377

§ 371 Date: Apr. 13, 2022

Related U.S. Application Data
(60) Provisional application No. 62/923,458, filed on Oct. 18, 2019.

(51) Int. Cl. G01N 33/543; A61K 49/00; A61L 26/00; A61L 27/18; A61L 27/54; A61L 27/56

(52) U.S. Cl. CPC G01N 33/54373; A61K 49/0056; A61K 49/0073; A61L 26/0061; A61L 27/18; A61L 27/54; A61L 27/56; A61L 2300/252; A61L 2430/24

(57) ABSTRACT

Provided herein is a biomaterial comprising a sensor system comprising a donor fluorophore linked to a target binding moiety (TBM) and an acceptor molecule linked to a TBM, wherein, when the TBM linked to the donor fluorophore and the TBM linked to the acceptor molecule binds to a target, a resonance energy transfer (RET; e.g., Forster (or Fluorescence) resonance energy transfer (FRET), bioluminescent resonance energy transfer (BRET), chemiluminescent resonance energy transfer (CRET), or a combination thereof) from the donor fluorophore to the acceptor molecule occurs and a detectable signal is produced. An medical device, e.g., an implant, comprising the presently disclosed biomaterial comprising a sensor system is further provided. Related medical devices and solid supports are furthermore provided herein. Use of the biomaterials and medical devices in methods of determining a level of expression of a gene, an RNA, or a protein, is additionally provided.

### CROSS-REFERENCE TO RELATED APPLICATIONS

This application claims the priority benefit under 35 U.S.C. § 119(e) of U.S. Provisional Patent Application No. 62/923,458, filed Oct. 18, 2019, which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “53563A_Seqlisting.txt”, which was created on Oct. 16, 2020 and is 2,013 bytes in size. The subject matter of the Sequence Listing is incorporated herein in its entirety by reference.

### BACKGROUND

Strategies for disease diagnosis, stratification, and monitoring are made possible by protein analysis. The protein levels of a given biomarker can also influence treatment decisions for patients diagnosed with a disease. For example, elevated levels of the protein Her2 can predict resistance to endocrine and chemotherapy treatment. In the clinic, proteins are assayed by a variety of tried and true techniques, including immunohistochemistry (IHC), enzyme linked-immunosorbent assay (ELISA), and flow cytometry, and many unique proteins are be detected by these methods. Powers and Palecek, J Healthc Eng 3(4): 503-534 (2012). However, researchers still seek ways to improve protein analysis in patients by increasing accuracy, sensitivity and specificity, decreasing the required sample size, and by reducing the invasiveness of the means for obtaining samples from the patient. For instance, Wang et al., Lab on a chip 11: 3411-3418 (2011) describes a simple and inexpensive microchip ELISA-based detection system for quantifying the ovarian cancer biomarker HE4 in urine samples. Also, Maeng et al., Biosens Bioelectron 23(9): 1319-1325 (2008) describes a microfluidic biosensor based on electrical detection of the cancer biomarker, alpha fetoprotein. Despite such efforts, there still remains a need for accurate, non-invasive protein assays that are capable of providing real-time in vivo protein levels.

### SUMMARY

Described herein for the first time are data demonstrating that a biomaterial comprising a resonance energy transfer (RET)-based sensor implanted into a subject may be used to provide accurate, real-time, in vivo target detection. In some aspects, the biomaterial comprises a FRET-based sensor system implanted into a subject that may be used to provide accurate, real-time, in vivo protein detection. The biomaterial was protein-specific and concentration-sensitive, as the readout signal increased as the detected protein level increased. Advantageously, the readout signal was obtained transcutaneously thereby meeting the non-invasiveness goal. The sensor system was programmable and could be made to detect virtually any protein and the biomaterial could be operated in different formats and therefore has the flexibility to work in a number of environments.

Accordingly, in some aspects the disclosure provides a biomaterial comprising a sensor system comprising a donor linked to a target binding moiety (TBM) and an acceptor molecule linked to a TBM, wherein, when the TBM linked to the donor and the TBM linked to the acceptor molecule binds to a target, a resonance energy transfer (RET) from the donor to the acceptor molecule (i) occurs or (ii) decreases or stops occurring and a detectable signal is produced. In any of the aspects or embodiments of the disclosure, the RET is Forster (or Fluorescence) resonance energy transfer (FRET), bioluminescent resonance energy transfer (BRET), chemiluminescent resonance energy transfer (CRET), or a combination thereof. In any of the aspects or embodiments of the disclosure, the donor is a donor fluorophore, a donor luminogenic protein, a donor chemiluminescent compound, or a combination thereof.

In some aspects, the present disclosure provides a biomaterial comprising a sensor system comprising a donor fluorophore linked to a target binding moiety (TBM) and an acceptor molecule linked to a TBM, wherein, when the TBM linked to the donor fluorophore and the TBM linked to the acceptor molecule binds to a target, a Forster resonance energy transfer (FRET) from the donor fluorophore to the acceptor molecule occurs and a detectable signal is produced.

The present disclosure also provides a medical device, e.g., implant, would dressing, comprising a presently disclosed biomaterial. In some aspects, the disclosure provides a solid support attached to a sensor system comprising a donor linked to a target binding moiety (TBM) and an acceptor molecule linked to a TBM, wherein, when the TBM linked to the donor and the TBM linked to the acceptor molecule binds to a target, a resonance energy transfer (RET) from the donor to the acceptor molecule (i) occurs or (ii) decreases or stops occurring and a detectable signal is produced, wherein the sensor system is in contact with a sample of a cell culture or a sample obtained from a subject. Also provided herein is a solid support attached to a sensor system comprising a donor fluorophore linked to a target binding moiety (TBM) and an acceptor molecule linked to a TBM, wherein, when the TBM linked to the donor fluorophore and the TBM linked to the acceptor molecule binds to a target, a Forster resonance energy transfer (FRET) from the donor fluorophore to the acceptor molecule occurs and a detectable signal is produced, wherein the sensor system is in contact with a sample of a cell culture or a sample obtained from a subject.

Without being bound to any particular theory, the presently disclosed biomaterials, medical devices, and solid supports are useful in methods of determining a level of expression of a gene, an RNA or a protein in a subject. In exemplary embodiments, the method comprises implanting into the subject a biomaterial of the present disclosure and detecting or measuring the detectable signal produced by the sensory system. In various aspects, the method determine the level of expression of a protein in a subject.

Methods of detecting a disease in a subject are further provided. In exemplary embodiments, the method comprises determining a level of expression of a gene, an RNA or a protein in a subject in accordance with the methods of the present disclosure which comprise using the presently disclosed biomaterials, medical devices, and solid supports.

The present disclosure also provides methods of monitoring progression, regression, or stage of a disease in a subject. In exemplary embodiments, the method comprises determining a level of expression of a gene, an RNA, or a protein, or a combination thereof, in a subject according to the methods of the present disclosure which comprise using the presently disclosed biomaterials, medical devices, and solid supports, at a first time point and at a second time point, wherein the expression level measured at the first time point is compared to the expression level measured at the second time point, wherein the difference in the level of expression at the second time point relative to the level of expression at the first time point is indicative of progression, regression, or stage of the disease.

The present disclosure furthermore provides methods of determining treatment for a subject with a disease. In exemplary embodiments, the method comprises determining a level of expression of a gene, an RNA, or a protein, or a combination thereof, in a subject according to the methods of the present disclosure which comprise using the presently disclosed biomaterials, medical devices, and solid supports, to determine a stage of a disease in the subject and determining the treatment for the subject based on the determined stage.

Methods of determining efficacy of a treatment for a disease in a subject are provided herein. In exemplary embodiments, the method comprises monitoring progression, regression, or stage of the disease in a subject in accordance the methods of the present disclosure before, during, and/or after treatment.

The present disclosure provides methods of treating a disease in a subject. In exemplary embodiments, the method comprises determining treatment for a subject with a disease in accordance with the presently disclosed methods of determining treatment and administering the treatment to the subject based on the outcome of the monitoring of the disease.

The present disclosure also provide methods of using the biomaterials, medical devices, and solid supports in an in vitro setting for purposes of determining the level of expression of a protein, e.g., by a cell culture, to determine the health of the cell culture.

### BRIEF DESCRIPTION OF THE DRAWINGS

FIG. 1A is a schematic of an exemplary method of making the biomaterials of the present disclosure. Target-Binding Moieties (TBM) are labeled with a donor fluorophore or an acceptor molecule to make Acceptor-Labeled TBMs and Donor-Labeled TBMs. Here, two TBMs each having a different structure but bind to the same target are shown. The Labeled-TBMs are reacted with a PEG (e.g., a 4-arm PEG) to create labeled TBM-PEG conjugates. Conjugates comprising more than one donor-labeled TBMs or more than one acceptor-labeled TBMs are made alongside conjugates comprising both donor-labeled TBMs and acceptor-labeled TBMs. Such conjugates comprising both types of labeled TBMs represent a sensor system. The sensor system is incorporated into a biomaterial. In exemplary aspects, the conjugates are mixed with unlabeled PEG and crosslinked. The boxed image on the right of FIG. 1A shows crosslinked arms of the PEG molecules. Some arms of the PEG are linked to an arm of another PEG molecule while other arms are linked to labeled TBMs. After incorporation into a biomaterial and implantation into the subject at an implantation site, the sensor system of the biomaterial is exposed to an in vivo environment wherein the target is absent or at low levels or is present or at high levels. The binding of the TBMs to the target allow for FRET from the donor to the acceptor to occur and the FRET signal may be detected. When target is absent or at lower levels, the target is not bound by both donor-labeled TBM and acceptor labeled TMB. And thus FRET from the donor to the acceptor does not occur.

FIG. 1B is a schematic similar to FIG. 1A, except that only one TBM (of same structure) is used in the sensor system. A batch of TBM1 is labeled with donor fluorophores and another batch of TBM1 is labeled with acceptor molecules. When reacted with PEG, conjugate species comprising a donor-labeled TBM1 and an acceptor-labeled TBM1 are made. When target is bound by both donor-labeled TBM1 and an acceptor-labeled TBM1, FRET from donor to acceptor occurs.

FIG. 1C is a schematic similar to FIG. 1B, except that only one TBM (of same structure) is used in the sensor system and a single batch of TBM1 is labeled with donor fluorophores and acceptor molecules. When reacted with PEG, the labeled TBM1 create conjugate species comprising TBM1 labeled with both acceptor and donor. When target is bound by donor- and acceptor-labeled TBM1, FRET from donor to acceptor occurs.

FIG. 1D is a schematic demonstrating different types of acceptor molecules. The TBMs bind to the target, FRET occurs from donor to acceptor and when an acceptor is a quencher, the detectable signal (e.g., fluorescence) at the donor wavelength decreases or is quenched, whereas, when the acceptor is an acceptor fluorophore that emits at a different wavelength than the donor wavelength, then the detectable signal (e.g., fluorescence) at the acceptor fluorophore wavelength increases. Also, the FRET signal (e.g., light signal) may be converted into a different type of signal, e.g., a radio wave or electric signal, and the signal may be sent to a device that provides a sensory alarm or a message.

FIG. 1E is a schematic of exemplary ways conjugates may be incorporated into a biomaterial. In one case, unlabeled PEG is added to the conjugates and then a crosslinker crosslinks the PEG and conjugates to form a gel. In another instance, the conjugates are applied to an implant, e.g., at the surface or within the pores of the implant, and the crosslinked in place. Also, if a biomaterial is functionalized with a chemical moiety then the free arms of the PEG may be functionalized with a chemical moiety which reacts with the chemical moiety of the biomaterial. Other means for attaching the conjugates to the biomaterial are contemplated and further described herein.

FIG. 1F is a schematic of various ways biomaterials comprise the sensor system. In exemplary instances, the biomaterial may encapsulated, house, contain the sensory system. In exemplary instances, the biomaterial may be impregnated, infused, or saturated with the sensor system. In various instances, the sensor system is attached to the surface of the biomaterial with or without a linker.

FIG. 2A is a graph of the signal intensity plotted as a function of VEGF-A concentration (ng/mL).

FIG. 2B is an image of the FRET signal of wells of a multi-well plate comprising a biomaterial comprising TBMs labeled with Cy5.5 only (top row) or labeled with Cy7 only (middle row) or comprising TBMs labeled with Cy5.5 and with TBMs labeled with Cy7 (bottom row) when incubated with a solution comprising various concentrations of VEGF-A.

FIG. 2C is an image of the FRET signals of wells of a multi-well plate comprising a biomaterial comprising a sensor system incubated with control media (lacking S100a9), a 1:1 ratio of control media and media obtained from a culture of splenocytes obtained from 4T1 tumor bearing mice, or with full strength media obtained from a culture of splenocytes obtained from 4T1 tumor bearing mice.

FIG. 3 is an image of the FRET signals of implanted hydrogels comprising a sensor system that were incubated with 0 ng or 1000 ng of VEGF-A prior to implantation.

FIG. 4 is a series of images of the FRET signals taken on Day 0, Day 1, or Day 5 post-implantation of implanted biomaterials comprising a sensor system doped with Dex, IFN-gamma or nothing.

FIG. 5A is an image of the FRET signals of Tumor-Free mice or Tumor-Bearing mice (boxed) comprising a biomaterial comprising a sensor system.

FIG. 5B is a graph of the normalized intensity of Tumor-Free (TF) mice or Tumor-Bearing (TB) mice.

FIG. 6 graph of the normalized intensity of a protein plotted as a function of time.

FIG. 7A shows a schematic of a DNA aptamer coupled to luciferase at one end and a quencher at the other end. The middle portion of the schematic depicts that upon target binding, the aptamer is opened and the luciferase becomes detectable due to the increase in distance of the luciferase from the quencher, thereby decreasing or stopping the RET (e.g., BRET) from occurring. The bottom portion of the schematic shows that such DNA aptamers coupled to luciferase and a quencher can be incorporated into an implantable hydrogel sensor or tethered to another material to detect a target. FIG. 7B shows that the DNA aptamer coupled to luciferase at one end and a quencher at the other end detected interferon-γ in solution.

FIG. 8 shows results of experiments utilizing the luciferase and quencher modified aptamer (as described in Example 14) attached to a 40 kDa PEG macromer. Interferon-γ is in labeled well 1, and the negative control is shown in labeled well 2. Labeled well 3 is a complementary DNA strand (positive control).

FIG. 9 shows results of experiments utilizing the luciferase and quencher modified aptamer (as described in Example 14) attached to a 40 kDa PEG macromer. Then the macromer was doped into a larger PEG hydrogel solution and the entire network was crosslinked. Interferon-γ is in labeled well 1, and the negative control is shown in labeled well 2. Labeled well 3 is a complementary DNA strand (positive control).

FIG. 10 depicts a schematic of a mesh-like structure with a sensor system impregnated, infused, or saturated into the structure. The mesh-like structure may be an implantable scaffold, a wound dressing, an outer porous surface coating of an orthopedic implant, or any other suitable implantable structure. As may be true with any of the figures, it is not drawn to scale, and the sensor material is depicted as dots for ease of illustration.

FIG. 11 depicts a schematic of a scaffold with a sensor system impregnated, infused, or saturated into the structure. The scaffold may be implanted in any suitable location of the body, including, for example, subcutaneously (e.g., in the fatty layer directly below the skin) in the arm or other easily interrogatable location.

FIG. 12 depicts a schematic of a detection system for detecting signals emitted from the sensor system. The detection system may comprise a photodetector and converting system for detecting a light signal and converting it to an electrical signal; it may also comprise a receiver for receiving, processing, and optionally, analyzing the electrical signal. Specifically, FIG. 12 depicts a schematic of an arm with a medical device (not visible) implanted therein. A watch or other device for detecting and converting light signals is provided along with a mobile phone or other computing device for receiving electrical signals transmitted by the converting system.

### DETAILED DESCRIPTION

Sensor System

The present disclosure relates to a sensor system for detecting and/or measuring a target. In any of the aspects or embodiments of the disclosure, the sensor system comprises a donor linked to a target binding moiety (TBM) and an acceptor molecule linked to a TBM. In any of the aspects or embodiments of the disclosure, the donor and acceptor molecule of the sensor system form a resonance energy transfer (RET) pair. RET systems contemplated by the disclosure include any non-radiative energy transfer system, such as Forster resonance energy transfer (FRET), bioluminescent resonance energy transfer (BRET), and chemiluminescent resonance energy transfer (CRET). Donors contemplated by the disclosure include without limitation a donor fluorophore, a donor luminogenic protein, a donor chemiluminescent (CL) compound, or a combination thereof. FRET, BRET and CRET exploit non-radiative energy transfer from an excited donor to an acceptor molecule in the ground-state when they are in close proximity (e.g., 1-10 nanometers).

BRET involves the use of a donor luminogenic protein that is an enzyme (e.g., a luciferase enzyme), and a substrate (e.g., luciferin) that interact to produce light. In BRET, the energy from the light is diverted into an acceptor molecule. The acceptor is, in various embodiments, a fluorophore which will then emit fluorescence, or a quencher which will not emit light.

CRET involves non-radiative energy transfer from a chemiluminescent (CL) donor compound to a fluorophore acceptor or quencher. CRET occurs by the oxidation of a CL compound that then excites the fluorescent acceptor. The oxidation is catalyzed, for example and without limitation, by a peroxidase enzyme. Such catalysis occurs, in various embodiments, in the presence of hydrogen peroxide.

In exemplary embodiments, the sensor system comprises a donor fluorophore linked to a target binding moiety (TBM) and an acceptor molecule linked to a TBM. In exemplary instances, the donor fluorophore and acceptor molecule of the sensor system form a Forster resonance energy transfer (FRET) pair, such that the donor fluorophore transfers energy to the acceptor molecule through non-radiative dipole-dipole coupling. In various aspects, the efficiency of the energy transfer is proportional to the distance between the donor fluorophore and the acceptor molecule. Without being bound to a particular theory, with regard to the presently disclosed sensor systems, FRET occurs from the donor fluorophore to the acceptor molecule, when the TBM linked to the donor fluorophore and the TBM linked to the acceptor molecule binds to a target, as binding to the target brings the donor fluorophore in a close proximity to the acceptor molecule so that FRET may occur with high efficiency. In various aspects, binding of the TBM linked to the donor fluorophore and the TBM linked to the acceptor molecule to the target brings the donor fluorophore within about 10 angstroms to about 100 angstroms of the acceptor molecule. When FRET from the donor fluorophore to the acceptor molecule occurs, a detectable signal is produced.

FRET and commonly used FRET pairs are known in the art. See, e.g., Bajar et al., Sensors (Basel) 16(9): 1488 (2016) and Sapsford et al., Angew Chem Int Ed 45: 4562-4588 (2006); the entirety of each is incorporated herein by reference. In various aspects, the absorption spectrum of the acceptor molecule overlaps with the fluorescence emission spectrum of the donor and/or the donor and acceptor transition dipole orientations are approximately parallel. In various aspects, the FRET pair comprises at least one fluorophore, e.g., a donor fluorophore. In various aspects, the FRET pair comprises two different fluorophores, e.g., a donor fluorophore and an acceptor fluorophore.

Fluorophores commonly used as FRET pairs include small organic dyes, fluorescent proteins (FPs), and quantum dots (Bajar et al., supra). In various aspects, the FRET pair comprises a small organic dye, e.g., Cy3, ATTO550, Alexa555 (as donors) and/or e.g., Cy5, ATT0647N, Alexa647 (as acceptors). The dye in certain aspects is a BODIPY dye, a Cy3 dye, a Cy5 dye, a Cy7 dye, a HiLyte Fluor dye, an ATTO Dye, an AlexaFluor dye, a DY Dye, naphthalene, pyrene, coumarin, fluorescein, rhodamine, cyanine, TAMRA, TMR, FITC, ROX, DyLight, Texas Red, BIDIPY, and the like. Additional dyes are taught in the prior art. See, e.g., Sapsford et al., supra, Roy et al., Nat Methods 5(6): 507-516 (2008). In various aspects, the donor fluorophore is fluorescein and the acceptor molecule is tetramethylrhodamine. In various aspects, the donor fluorophore is IAEDANS and the acceptor molecule is fluorescein. In various aspects, the donor fluorophore is EDANS and the acceptor molecule is Dabcyl. In various aspects, the donor fluorophore is BODIPY FL and the acceptor molecule is BODIPY FL. fluorescein. In various aspects, the donor fluorophore is fluorescein and the acceptor molecule is QSY 7 or a QSY 9 dye. In various aspects, the donor fluorophore is Alexa Fluor 350 and the acceptor molecule is Alexa Fluor 488. In various aspects, the donor fluorophore is Alexa Fluor 488 and the acceptor molecule is Alexa Fluor 546, Alexa Fluor 555, Alexa Fluor 568, Alexa Fluor 594, or Alexa Fluor 647. In various aspects, the donor fluorophore is Alexa Fluor 546 and the acceptor molecule is Alexa Fluor 568, Alexa Fluor 594, or Alexa Fluor 647. In various aspects, the donor fluorophore is Alexa Fluor 555 and the acceptor molecule is Alexa Fluor 594, or Alexa Fluor 647. In various aspects, the donor fluorophore is Alexa Fluor 568 and the acceptor molecule is Alexa Fluor 647.

Donor luminogenic proteins commonly used in BRET include, without limitation, Firefly luciferase, Gaussia Luciferase, Renilla Luciferase, aequorin, and click-beetle luciferase, Oplophorus luciferase. The disclosure also contemplates the use of variants and analogs of proteins described herein, including luminogenic proteins. As used herein a “variant” refers to a protein or analog thereof that is modified to comprise additional chemical moieties (e.g., glycosylation, pegylation, and/or polysialylation) not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it. Fragments of luminogenic proteins are also contemplated herein.

Donor chemiluminescent compounds contemplated by the disclosure include, without limitation, luminol.

In exemplary aspects, the FRET pair comprises a fluorescent protein (FP) pair, e.g., an enhanced blue FP (EBFP)-enhanced green FP (EGFP) FRET pair, a blue FP (BFP)-green FP (GFP) FRET pair, a cyan FP (CFP)-yellow FP (YFP) FRET pair, or a green fluorescent protein (GFP)-red fluorescent protein (RFP) FRET pair. In various instances, the FRET pair comprises one or more of the following CFP/YFP: aquamarine; enhanced CFP, mTurquoise2, mCerulean3, LUMP^{m}, mTFP1, enhanced YFP, mVenus, sEYFP, mCitrine, or YPet. In various instances, the FRET pair comprises one or more of the following GFP/RFP: EGFP, NowGFP, Clover, mClover3, mNeonGreen, mRuby2, mRuby3, mCherry. In various instances, the FRET pair comprises one or more of the following far-red FP (FFP)/infrared FP (IFP): mPlum, eqFP650, mCarinal, IFP1.4^{m}, iRFP^{m}. In various instances, the FRET pair comprises one or more of the following large Stokes shift (LSS) FPs and FP acceptors: mAmetrine, LSSmOrange, tdTomato, mKate2. In various instances, the FRET pair comprises one or more of the following Dark FPs: ShadowG, REACh1, REACh2, sREACh. In various instances, the FRET pair comprises one or more of the following phototransformable FPs: rsTagRFP, PA-GFP, Phanta. In various instances, the FRET pair comprises one or more of the following FPs for multicolor FRET: T-Sapphire, mTagBFP, sfGFT, CyOFP1, mOrange2, mKOK, TagRFP, DsRed. In various aspects, the FRET pair is one of the following pairs: ECFP-EYFP, mTurquoise2-sEYFP, mTurquoise2-mVenus, EGFP-mCherry, Clover-mRuby2, mClover3-mRuby3, mNeonGreen-mRuby3, eqFP650-iRFP, mAmetrine-tdTomato, LSSmOrange-mKate2, EGFP-SREACh, EGFP-ShadowG, EGFP-activated PA-GFP, EGFP-Phanta, mTagBFP-sfGFP, mVenus-MKOK, CyOFP1-mCardinal. Such FRET pairs are described in Bajar et al., supra.

In exemplary instances, the acceptor molecule is a quencher. Quenchers are known in the art and include, e.g., DNP, DABCYL, DABSYL, QXL 490, QSY, ATTO, QSY9, BHQ1, QSY21, ATTO612Q, BHQ3, QXL 670, BBQ 650, Cy5Q, Cy7Q. In various aspects, the donor fluorophore is Alexa Fluor 350 and the quencher is QSY 35 or dabcyl. In various aspects, the donor fluorophore is Alexa Fluor 488 or Alexa Fluor 546 and the quencher is QSY 35 or dabcyl or QSY 7 and QSY 9. In various aspects, the donor fluorophore is Alexa Fluor 555 and the quencher is QSY 7 and QSY 9. In various instances, the donor fluorophore is Alexa Fluor 568 and the quencher is dabcyl or QSY 7 and QSY 9. In various instances, the donor fluorophore is Alexa Fluor 594 or Alexa Fluor 647 and the quencher is QSY 7 and QSY 9.

Fluorophores are commercially available through ThermoFisher Scientific (Waltham, MA), Premier Biosoft (Palo Alto, CA), Lumiprobe (Hunt Valley, MD), and Enzo Life Sciences (Farmindale, NY).

Targets and Target Binding Moieties (TBMs)

In various aspects, the sensor system comprises one or more structurally distinct Target Binding Moieties (TBMs). In various aspects, the sensory system is specific to one target, e.g., the sensor system detects and/or measures only one target. In various aspects, all of the TBMs of the sensor system bind to the same target. The sensor system in exemplary aspects comprises multiple structurally distinct TBMs to detect and/or measure multiple targets. In various aspects, the sensor system produces a distinct detectable signal for each and every unique target. In various instances, a subset of the TBMs of the sensor system binds to a first target and at least one other subset of the TBMs of the sensor system binds to a second target, wherein a first detectable signal is produced when the first target is bound and a second detectable signal is produced when the second target is bound, wherein the first detectable signal is distinct from the second detectable signal. Optionally, the TBMs of the sensor system collectively bind to three or more targets, wherein a distinct detectable signal is produced for each target. In various aspects, the TBMs of the sensor system collectively bind to 5 to 10 targets, wherein, for each target, a distinct detectable signal is produced upon binding of the TBMs to its target. In exemplary aspects, the TBMs of the sensor system collectively bind to more than 10, more than 25, or more than 50 targets, wherein, for each target, a distinct detectable signal is produced upon binding of the TBMs to its target.

The TBM in various aspects is a peptide, polypeptide, or protein. The term “polypeptide” as used herein includes oligopeptides and refers to a single chain of amino acids connected by one or more peptide bonds. By “protein” is meant a molecule comprising one or more polypeptide chains. The protein of the in some aspects comprises, for example, 1, 2, 3, 4, 5, or more polypeptide chains. The term “peptide” generally refers to a single chain of amino acids connected by one or more peptide bonds wherein the number of amino acids of the peptide are typically less than about 100 amino acids. Optionally, the peptide is less than about 100 amino acids in length, optionally, less than about 75 amino acids in length.

The TBM in various aspects is an aptamer. The term “aptamer” refers to short polymer sequences (e.g., oligonucleic acid or peptide molecules) with high affinity and specificity for a given target. SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For more on aptamers, see, generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety. In various aspects, the disclosure contemplates use of a TBM that is a dual labeled aptamer, wherein a donor is attached at one end of the TBM aptamer and an acceptor is attached to the other end of the TBM aptamer. In some embodiments, the donor is a donor luminogenic protein and the acceptor is a quencher. In various embodiments, the acceptor molecule in BRET is a quencher that quenches the fluorescence of the donor luminogenic protein when the TBM aptamer is not bound to the target. Upon target binding to the TBM aptamer, the TBM aptamer changes conformation such that the donor luminogenic protein is extended away from the quencher and luminescence is detectable.

In various aspects, the TBM is an antibody or an antigen-binding fragment thereof, optionally, a Fab fragment, or the TBM is an antibody protein product, optionally, a nanobody, a camelid, or an scFv. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

In various aspects, the antibody can be a monoclonal antibody or a polyclonal antibody. In some aspects, the antibody comprises a sequence that is substantially similar to a naturally-occurring antibody produced by a mammal, e.g., mouse, rat, rabbit, goat, horse, chicken, hamster, pig, human, and the like. In this regard, the antibody may be considered as a mammalian antibody, e.g., a mouse antibody, rat antibody, rabbit antibody, goat antibody, horse antibody, chicken antibody, hamster antibody, pig antibody, human antibody, and the like. In certain aspects, the recombinant protein is a monoclonal human antibody.

In certain aspects, the TBM is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

An antibody, in various aspects, is cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment.

The architecture of antibodies has been exploited to create a growing range of alternative antibody formats that spans a molecular-weight range of at least or about 12-150 kDa or more and a valency (n) range from monomeric (n=1), dimeric (n=2) and trimeric (n=3) to tetrameric (n=4) and potentially higher; such alternative antibody formats are referred to herein as “antibody protein products”.

Antibody protein products can be an antigen binding format based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BslgG, appended IgG, BsAb fragments, bispecific fusion proteins and BsAb conjugates. See, e.g., Spiess et al., Molecular Immunology 67(2) Part A: 97-106 (2015).

In exemplary aspects, the TBM comprises any one of these antibody protein products (e.g., scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate).

The TBM may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

The choice of type of TBM in various aspects depends on the target. In exemplary aspects, the target is a protein and the TBM is a peptide, polypeptide, protein, aptamer, antibody, antigen binding fragment thereof, antibody protein product, including any of those described above.

In exemplary instances, the target is a gene or gene product, e.g., an RNA encoded by the gene or a protein encoded by the gene. In exemplary aspects, when the target comprises a nucleic acid, then the TBM comprises a nucleic acid which binds to the target. By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

The TBMs are linked to a donor (e.g., donor fluorophore, donor luminogenic protein, donor chemiluminescent molecule) and/or an acceptor molecule. The TBMs in various aspects are linked by covalent bonds (e.g., a peptide, ester, amide, or sulfhydryl bond) or non-covalent bonds (e.g., via hydrophobic interaction, hydrogen bond, van der Waals bond, electrostatic or ionic interaction), or a combination thereof. In various embodiments, the TBMs are chemically modified with various substituents or moieties that permit covalent linkages to the donor (e.g., donor fluorophore, donor luminogenic protein, donor chemiluminescent molecule) and/or acceptor molecule. In various aspects, when the TBM is a peptide, polypeptide, or a protein, the donor (e.g., donor fluorophore, donor luminogenic protein, donor chemiluminescent molecule) and/or acceptor molecule may be attached at the N- or C-terminus of the TBM via the amine or carboxyl groups. Side chains of amino acids of the TBMs also permit covalent linkage to donor (e.g., donor fluorophore, donor luminogenic protein, donor chemiluminescent molecule) and/or acceptor molecule via side chain moieties. In some embodiments, a donor and an acceptor molecule are attached to the same TBM.

Cysteinyl residues most commonly are reacted with haloacetates (and corresponding amines), such as chloroacetic acid or chloroacetamide, to give carboxymethyl or carbocyamidomethyl derivatives. Cysteinyl residues also are derivatized by reaction with bromotrifluoroacetone, α-bromo-.beta.(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylissurea; 2,4 pentanedione; and transaminase catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R1) such as 1-cyclohexyl-3-(2-morpholinyl-(4-ethyl) carbodiimide or 1-ethyl-3 (4 azonia 4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues may be used in attaching the donor (e.g., donor fluorophore, donor luminogenic protein, donor chemiluminescent molecule) and/or the acceptor molecule.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecule Properties, W. H. Freeman & Co., San Francisco, pp. 79-86, 1983), acetylation of the N-terminal amine, and, in some instances, amidation of the C-terminal carboxyl groups. Such derivatives are chemically modified polypeptide compositions in which the binding construct polypeptide is linked to a polymer.

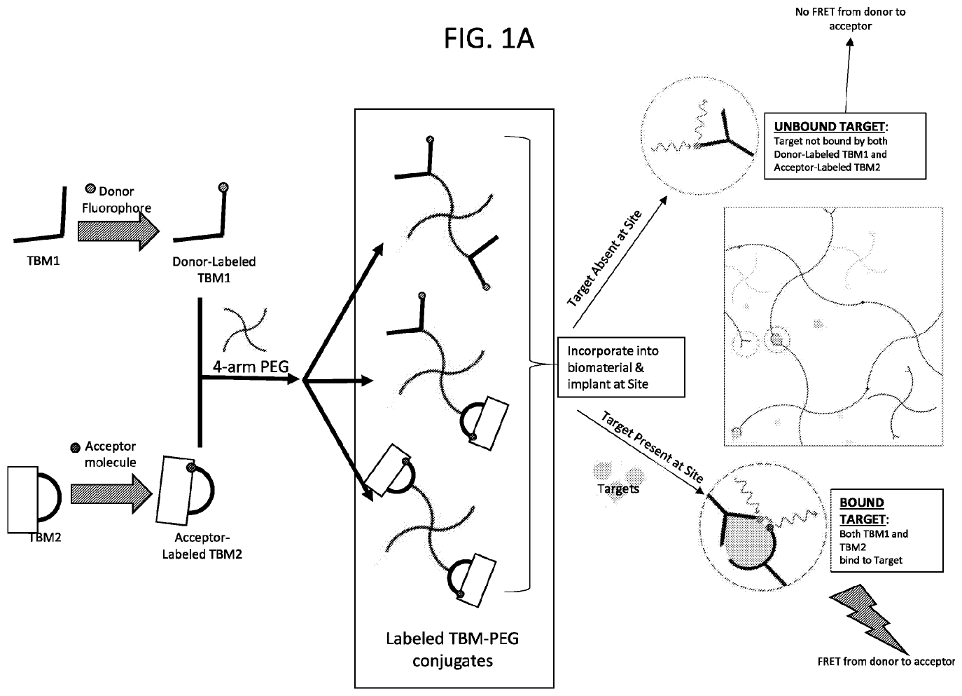

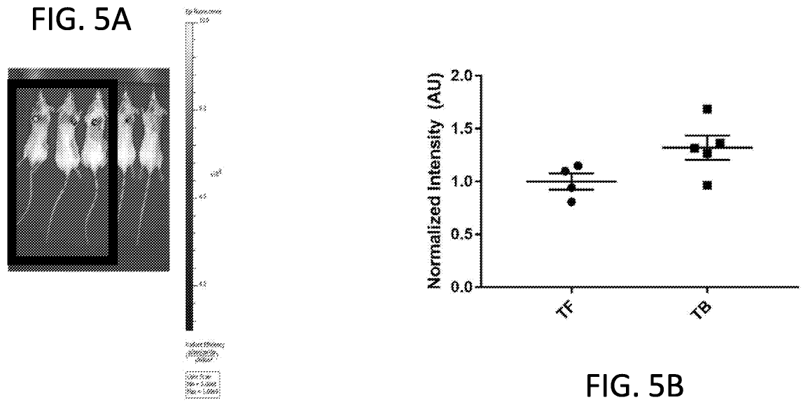

In various instances, the TBM linked to the donor (e.g., donor fluorophore, donor luminogenic protein, donor chemiluminescent molecule) is also linked to the acceptor molecule. FIG. 1C provides a schematic of such a dual labeled TBM. In some embodiments, a dual labeled TBM is an aptamer. In some embodiments, a dual labeled TBM is a DNA aptamer comprising an acceptor molecule (e.g., quencher) attached at one end of the DNA aptamer and a donor attached at the opposite end (see, e.g., FIG. 7A) of the DNA aptamer. In various embodiments, and as described herein, the donor is a donor fluorophore, a donor luminogenic protein, a donor chemiluminescent compound, or a combination thereof. In various aspects, the sensor system comprises a first TBM linked to the donor (e.g., donor fluorophore, donor luminogenic protein, donor chemiluminescent molecule) and a second TBM linked to the acceptor molecule. In various aspects, the first TBM and the second TBM are structurally the same. In alternative instances, the first TBM is structurally distinct from the second TBM or the first TBM and the second TBM are structurally different. For instance both are peptides but different in amino acid sequences. In exemplary instances, the first TBM binds to a binding site on the target distinct from the binding site of the second TBM.

In various aspects, the acceptor molecule comprises an acceptor fluorophore which emits fluorescence at a wavelength different from the wavelength at which the donor fluorophore emits fluorescence, and the detectable signal is fluorescence emitted at the acceptor fluorophore wavelength. Optionally, each of the acceptor fluorophore and donor fluorophore emits fluorescence at a wavelength within a range of about 550 nm to about 900 nm, optionally, about 650 nm to about 870 nm. Further description of suitable acceptor fluorophores are provided herein. In various aspects, the acceptor molecule comprises a quencher moiety which quenches the fluorescence emitted by the donor fluorophore, and the detectable signal is a quenched fluorescence at the wavelength at which the donor fluorophore emits fluorescence. Optionally, the quencher comprises a metal ion or a quencher fluorophore. In some embodiments, the quencher comprises an inhibitor of enzyme activity.

Targets

The presently disclosed sensor systems are not limited to any particular target. Accordingly, the TBM may bind to virtually any target. In exemplary aspects, the TBM binds to a hormone, growth factor, cytokine, a cell-surface receptor, tumor antigen, or any ligand thereof. In exemplary aspects, the TBM binds to a protein expressed on the cell surface of a cell, e.g., an immune cells or a cancer cell. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein Ill, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is p53, KRAS, NRAS, MAGEA, MAGEB, MAGEC, BAGE, GAGE, LAGE/NY-ESO1, SSX, tyrosinase, gp100/pmeli7, Melan-A/MART-1, gp75/TRP1, TRP2, CEA, RAGE-1, HER2/NEU, WT1.

Strictly for purposes of exemplifying the different targets, the TBM may bind to any of the following cluster of differentiation molecules: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD79p, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the TBM is an antibody which is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor 1), U.S. Pat. Nos. 7,566,772 and 7,964,193 (interleukin-1β), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the TBM is an antibody which is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), l'Ibritumomab tiuxetan (product marketed with the brand name Zevalin®), l'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), l'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab. In exemplary aspects, the antibody binds to a tumor associated antigen and is an anti-cancer antibody. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies, such as siltuximab; and anti-VEGF antibodies such as bevacizumab; anti-IL6 receptor antibodies such as tocilizumab.

In various aspects, the targets are gene products of genes of a gene expression signature (GES) indicative or predictive of a disease or a medical condition, or risk therefor, optionally, wherein the gene products are proteins.

The disease or medical condition may be an autoimmune disease, inflammatory disease, cancer, metabolic disease, cardiovascular disease, kidney disease, wound healing, transplant rejection, allergy, or aging. Also, for example, the disease or medical condition is diabetes, inflammation, multiple sclerosis (MS), transplant rejection, or cancer. Optionally, the target are gene products of genes of a GES for metastatic cancer. Optionally, the target are gene products of genes of a GES for RR-MS.

Biomaterials

The present disclosure provides a biomaterial comprising a presently disclosed sensor system. In various aspects, the sensor system is covalently attached to the biomaterial. Optionally, the sensor system is attached to the biomaterial via non covalent bonds. In exemplary instances, the sensor system is encapsulated or housed by the biomaterial. In certain aspects, the sensor system is impregnated, saturated or infused throughout the biomaterial. For example, the biomaterial is porous and the sensor system is located in or impregnated in the pores of the biomaterial.

As used herein, the term “biomaterial” refers to any synthetic or natural material suitable for use in making artificial organs and prostheses or to replace bone or tissue. In exemplary embodiments, the biomaterial has been engineered to interact with biological systems for a medical purpose—either a therapeutic or a diagnostic one. In various aspects, the biomaterial comprises a polymer, metal, ceramic or composite material. Suitable polymers are known in the art. See, e.g., Wei et al., Int J Biomaterials 2018 Article 7158621. The polymer may be branched or unbranched. The polymer may be of any molecular weight. The polymer in some embodiments has an average molecular weight of between about 2 kDa to about 100 kDa (the term “about” indicating that in preparations of a water soluble polymer, some molecules will weigh more, some less, than the stated molecular weight). The average molecular weight of the polymer is in some aspect between about 5 kDa and about 50 kDa, between about 12 kDa to about 40 kDa or between about 20 kDa to about 35 kDa.

In various embodiments, the polymer is modified to have a single reactive group, such as an active ester for acylation or an aldehyde for alkylation, so that the degree of polymerization may be controlled or that the polymer may be linked to another moiety. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer.

In some embodiments, the polymer is selected from the group consisting of: Polytetra fluro ethylene (PTFE) or expanded PTFE (ePTFE), polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion.

In some aspects, the polymer is a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof. In some aspects, the water soluble polymers or mixtures thereof include, but are not limited to, N-linked or O-linked carbohydrates, sugars, phosphates, carbohydrates; sugars; phosphates; polyethylene glycol (PEG) (including the forms of PEG that have been used to derivatize proteins, including mono-(C1-C 10) alkoxy- or aryloxy-polyethylene glycol); monomethoxy-polyethylene glycol; dextran (such as low molecular weight dextran, of, for example about 6 kD), cellulose; cellulose; other carbohydrate-based polymers, poly-(N-vinyl pyrrolidone)polyethylene glycol, propylene glycol homopolymers, a polypropylene oxide/ethylene oxide co-polymer, polyoxyethylated polyols (e.g., glycerol) and polyvinyl alcohol.

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophibic nature, thus creating a water shell or hydration sphere when attached to other proteins or polymer surfaces. PEG is nontoxic, non-immunogenic, and approved by the Food and Drug Administration for internal consumption. In various aspects, the PEG is 3-arm PEG, 4-arm PEG, or 8-arm PEG. Optionally, at least one arm of the PEG is linked to a TBM linked to an acceptor molecule and/or a donor (e.g., donor fluorophore, donor luminogenic protein, donor chemiluminescent molecule). In some aspects, two or more arms of the PEG are linked to a TBM linked to an acceptor molecule and/or a donor (e.g., donor fluorophore, donor luminogenic protein, donor chemiluminescent molecule). In exemplary aspects, at least one arm of the PEG is linked to an arm of another PEG.

Optionally, the biomaterial of the present disclosure comprises a crosslinked polymer. In various aspects, the biomaterial is a gel optionally a hydrogel. Optionally, the biomaterial further comprises a therapeutic agent, optionally, wherein the therapeutic agent is released from the biomaterial.

In various aspects, the biomaterial is a metallic biomaterial, which optionally comprises titanium, platinum, gold, or an alloys of the foregoing. In various aspects, the metallic biomaterial comprises nitinol, chromium, cobalt, magnesium, or an alloy thereof.

In exemplary instances, the biomaterial is a ceramic biomaterial, optionally, one which comprise hydroxyapatite and/or Al₂O₃.

In various instances, the biomaterial comprises one or more naturally derived materials including but not limited to gelatin, collagen, hyaluronan, alginate, chitosan, chitin, and the like.

In exemplary aspects, the biomaterial comprises one or more decellularized materials, including but not limited to decellularized skin (i.e. strattice, alloderm); decellularized SIS, bladder, heart, muscle, brain, or hydrogels derived from the same.

Medical Devices

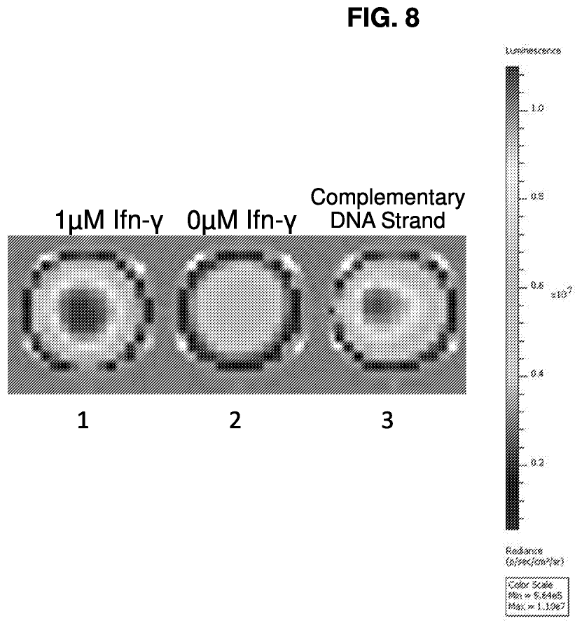

The present disclosure provides a medical device comprising a presently disclosed sensor system, optionally, a medical device comprising a presently disclosed biomaterial comprising a sensor system. As used herein, the term “medical device” refers to any device considered by the U.S. Food and Drug Administration (FDA) as a medical device and is listed in the CDRH classification database accessible at https://www.accessdata.fda.gov/scripts/cdrh/cfdocs/cfPCD/classification.cfm. The medical device in various aspects is an implantable medical device, which is also referred to herein as an “implant” which device is placed in the body of a subject. An implant may be placed in any part of the body of a subject. In exemplary aspects, the implant is placed in the epidermis or dermis of the subject, a body cavity, or a body orifice, or within tissue, e.g., muscle tissue, or inside or adjacent to an organ. The placement of the implant in exemplary instances allows for body fluids to pass near or over the medical device for detection and/or measurement of one or more targets. In various instances, the implant is a polymeric scaffold, or a prosthetic implant, e.g., a hip implant. The implant may be any of orthopedic implant, e.g., including rods, wires, plates, and screws. In various aspects, the implant is a biomaterial implant, a prosthetic device, a hernia mesh, a wound dressing, a cosmetic implant, any implant designed to promote growth and repair or regeneration of tissue, and the like. In various instances, the medical device, e.g., implant, comprise a metal and/or a ceramic material and/or polymer. In various aspects, the implant comprises a plurality of micropores, optionally, about 250 μm to about 700 μm. In various aspects, the implant is a biomaterial, optionally, a gel, is adhered to the surface of the implant. In various aspects, the medical device comprise a biomaterial optionally a gel and the biomaterial is present in the pores of the implant. Optionally, the biomaterial is present only in the pores of the implant. In various instances, the TBMs are located at a unique location of the implant, wherein detection of the detectable signal at the unique location enables identification of the target bound by the TBMs. In various aspects, the medical device comprises a porous or mesh-like structure, such as that depicted in FIG. 10 with one or more sensor systems coupled to and/or present within or on the porous or mesh-like structure. The one or more sensor systems may be impregnated, infused, or saturated into the mesh-like structure. The sensor system may reside within the pores of the mesh-like structure. In some aspects, the entire medical device may be porous or mesh-like; in other aspects, surface features of the medical device may be porous or mesh-like.

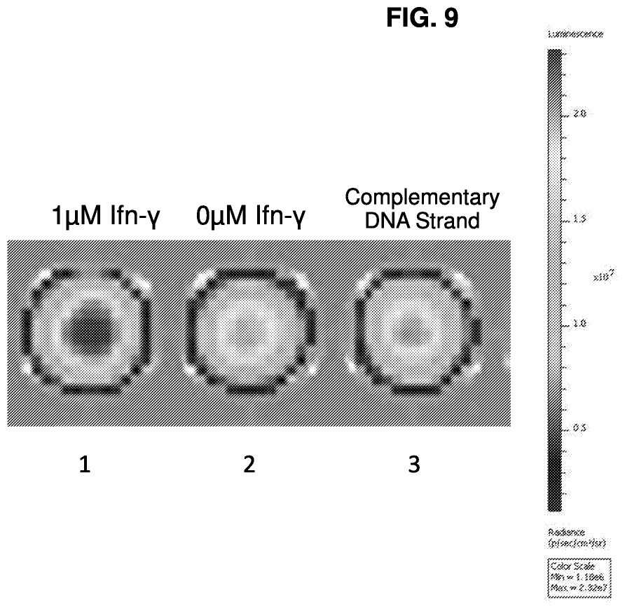

In various aspects of the foregoing, the medical device is a polymeric medical device. Optionally, the medical device is a naturally-derived medical device. In some aspects, the medical device is a wound dressing. In various aspects, the implantable medical device is a scaffold, such as, for example, the scaffold depicted in FIG. 11. In various aspects, the scaffold is one described in International Patent Publication No. WO2019/071257, the entire contents of which is incorporated herein by reference. In exemplary aspects, the scaffold is an implant intended for residence in tissue for several weeks to years and facilitates ingrowth of tissue. Such scaffolds are known in the art. See, e.g., Azarin et al., Nat Commun 6: 8094 (2015); Aguado et al., Sci Rep 5: 17566 (2015); Aguado et al., Acta Biomaterialia (2016); and Rao et al., Cancer Res 76(18): 5209-5218 (2016); U.S. Patent Application Publication No. 2014/0072510 A1; International Patent Application Publication No. WO 2017/120486.

In exemplary embodiments, the scaffold is partially or exclusively composed of a micro-porous poly(e-caprolactone) (PCL), forming a PCL scaffold. Such PCL scaffolds have a greater stability than the micro-porous poly(lactide-co-glycolide) (PLG) biomaterial scaffolds. In exemplary embodiments, the scaffold comprises PCL and/or PEG and/or alginate.

In some embodiments, the scaffold is a controlled release scaffold formed partially or exclusively of hydrogel, e.g., a poly(ethylene glycol) (PEG) hydrogel to form a PEG scaffold. Any PEG is contemplated for use in the compositions and methods of the disclosure. In general, the PEG has an average molecular weight of at least about 5,000 daltons. In further embodiments, the PEG has an average molecular weight of at least 10,000 daltons, 15,000 daltons, and is preferably between 5,000 and 20,000 daltons, or between 15,000 and 20,000 daltons. Also preferred is PEG having an average molecular weight of 5,000, of 6,000, of 7,000, of 8,000, of 9,000, of 10,000, of 11,000, of 12,000 of 13,000, of 14,000, or of 25,000 daltons. In further embodiments, the PEG is a four-arm PEG. In preferred embodiments, each arm of the four-arm PEG is terminated in an acrylate, a vinyl sulfone, or a maleimide. It is contemplated that use of vinyl sulfone or maleimide in the PEG scaffold renders the scaffold resistant to degradation. It is further contemplated that use of acrylate in the PEG scaffold renders the scaffold susceptible to degradation.

In exemplary embodiments, the scaffold is porous and/or permeable. In exemplary embodiments, the scaffold comprises a polymeric matrix and acts as a substrate permissible for metastasis, colonization, cell growth, etc. In exemplary embodiments, the scaffold provides an environment for attachment, incorporation, adhesion, encapsulation, etc. of agents (e.g., DNA, lentivirus, protein, cells, etc.). In exemplary embodiments, agents are released (e.g., controlled or sustained release) to attract cells, e.g., circulating tumor cells, metastatic cells, or pre-metastatic cells. The scaffolds in certain embodiments provide a sustained release depot formulation with the following non-limiting characteristics: (1) the process used to prepare the matrix does not chemically or physically damage the agent; (2) the matrix maintains the stability of the agent against denaturation or other metabolic conversion by protection within the matrix until release, which is important for very long sustained release; (3) the entrapped agent is released from the hydrogel composition at a substantially uniform rate, following a kinetic profile, and furthermore, a particular agent can be prepared with two or more kinetic profiles, for example, to provide in certain embodiments, a loading dose and then a sustained release dose; (4) the desired release profile can be selected by varying the components and the process by which the matrix is prepared; and (5) the matrix is nontoxic and degradable. PEG scaffolds as disclosed herein are also contemplated to function as a scaffold that achieves sustained release of a therapeutically active agent. Accordingly, in some embodiments an agent is configured for specific release rates. In further embodiments, multiple different agents are configured for different release rates. For example, a first agent may release over a period of hours while a second agent releases over a longer period of time (e.g., days, weeks, months, etc.). In some embodiments, and as described above, the scaffold or a portion thereof is configured for sustained release of agents. In some embodiments, the sustained release provides release of biologically active amounts of the agent over a period of at least 30 days (e.g., 40 days, 50 days, 60 days, 70 days, 80 days, 90 days, 100 days, 180 days, etc.).

In various instances, the medical device comprises the sensor system in the absence of a biomaterial. In some aspects, the medical device is functionalized for chemical attachment of the TBMs which are in turn linked to the acceptor molecule and/or donors (e.g., donor fluorophore, donor luminogenic protein, donor chemiluminescent molecule). Accordingly, the present disclosure provides a medical device comprising a sensor system comprising a donor (e.g., donor fluorophore, donor luminogenic protein, donor chemiluminescent molecule) linked to a TBM and an acceptor molecule linked to a TBM, wherein, when the TBM linked to the donor (e.g., donor fluorophore, donor luminogenic protein, donor chemiluminescent molecule) and the TBM linked to the acceptor molecule binds to a target, a resonance energy transfer (RET) from the donor (e.g., donor fluorophore, donor luminogenic protein, donor chemiluminescent molecule) to the acceptor molecule occurs and a detectable signal is produced. In various aspects, the RET is FRET, BRET, CRET, or a combination thereof.

In various aspects, the medical device comprises a metal, plastic, glass, fabric, or silicon. Optionally, the metal is titanium. In some aspects, the solid support is a silicon wafer.

Solid Supports

The present disclosure provides a solid support comprising a presently disclosed sensor system, optionally, a solid support comprising a biomaterial comprising the sensor system. In exemplary aspects, the solid support is attached to a sensor system comprising a donor (e.g., donor fluorophore, donor luminogenic protein, donor chemiluminescent molecule) linked to a target binding moiety (TBM) and an acceptor molecule linked to a TBM, wherein, when the TBM linked to the donor (e.g., donor fluorophore, donor luminogenic protein, donor chemiluminescent molecule) and the TBM linked to the acceptor molecule binds to a target, a resonance energy transfer (RET) from the donor (e.g., donor fluorophore, donor luminogenic protein, donor chemiluminescent molecule) to the acceptor molecule occurs and a detectable signal is produced. In various embodiments, the RET is FRET, BRET, CRET, or a combination thereof. In various aspects, the sensor system is attached to the solid support via a biomaterial. For instance, the sensor system is linked to a biomaterial which is a gel optionally a hydrogel, as further described herein and the biomaterial is attached to the solid support via non-covalent or covalent bonds. In various aspects, the solid support does not comprise a biomaterial and the solid support is functionalized for chemical attachment of the TBMs of the sensor system, which TBMs are in turn linked to the acceptor molecule and/or donors (e.g., donor fluorophore, donor luminogenic protein, donor chemiluminescent molecule).

In various aspects, the solid support comprises a metal, plastic, glass, fabric, or silicon. Optionally, the metal is titanium. In some aspects, the solid support is a silicon wafer or is tissue culture plasticware.

In various embodiments, the sensor system is in contact with a sample of a cell culture or a sample obtained from a subject, e.g., biopsy. In exemplary aspects, the cell culture may be a cell culture purposed for recombinant protein production, e.g., by bacteria, yeast, mammalian cells, etc.). In various aspects, the cell culture is a stem cell culture purposed for regenerative medicine. The stem cells may be embryonic, mesenchymal, induced and the like. The cell culture may be for expanding or differentiating stem cells. In various instances, the cell culture may be for immunotherapy and the sensor system detects or measures proteins involved in expanding, engineering, or priming immune cells for immunotherapy. In various instances, the sensor system detects or measures proteins involved in monitoring tissue engineered constructs and the production or maintenance of the extracellular matrix. The cell culture may be for screening different drugs and monitoring the biological response thereto.

In various aspects of the above, the solid support or medical device or biomaterial comprises one or more electrodes. In some aspects, the solid support is functionalized with a moiety and the sensor system is covalently attached to the solid support via the moiety.

Detectable Signals

In various embodiments, when the target is bound by the TBM(s), a detectable signal is produced. In some aspects, when the TBM linked to the donor fluorophore and the TBM linked to the acceptor molecule bind to the target, RET occurs. In some embodiments, when the TBM linked to the donor luminogenic protein and the TBM linked to the acceptor molecule (e.g., a fluorophore) bind to the target, BRET occurs. In various aspects, when the TBM linked to the donor fluorophore and the TBM linked to the acceptor molecule bind to the target, FRET occurs. In exemplary aspects, the detectable signal is provided by the donor fluorophore or the acceptor fluorophore. Detection of the change in fluorescence emission at the donor wavelength to the acceptor wavelength in exemplary aspects provides the detectable signal. In some aspects, an increase in fluorescence at the acceptor fluorophore emission wavelength provides a detectable signal. In various instances, when the acceptor molecule is a quencher, the detectable signal is a decrease in the fluorescence emission at the donor fluorophore emission wavelength.

Also, contemplated herein are modified sensor systems wherein target binding reduces or halts FRET from donor fluorophore to acceptor molecule. In various aspects, the TBM linked to the donor fluorophore binds to the TBM linked to the acceptor molecule and FRET occurs, and when one of the TBMs binds to the target, FRET from the donor fluorophore to acceptor molecule decreases or ceases altogether. Both types of sensor systems and detectable signals are provided by the present disclosure. In some aspects, the disclosure provides modified sensor systems wherein target binding reduces or halts BRET from donor luminogenic protein to acceptor molecule. In various aspects, the TBM linked to the donor luminogenic protein and the TBM linked to the acceptor molecule (e.g., quencher) are part of a single molecule such as a dual labeled aptamer (e.g., a dual labeled DNA aptamer) and BRET occurs in the absence of target binding, and binding of the dual labeled aptamer to the target causes a conformational change in the dual labeled aptamer which separates the donor luminogenic protein and the acceptor molecule, and BRET from the donor luminogenic protein to acceptor molecule decreases or ceases altogether thereby producing a detectable signal in the form of luminescence at the emission wavelength of the donor luminogenic protein.

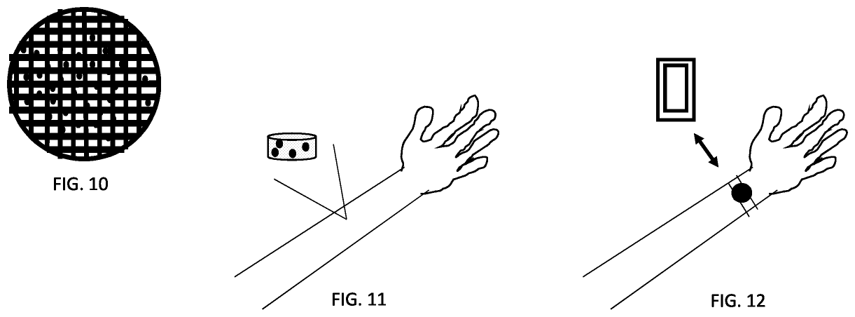

In various aspects, the detectable signal is detectable by an optical camera. Optionally, the detectable signal is detectable by a photosensor or photodetector. In some aspects, the sensor system further comprises a converting system which converts the FRET from the donor fluorophore to the acceptor molecule into an electrical signal. Optionally, the converting system comprises a photodetector which converts the light signal to current. In some aspects, the converting system has one or more processors and one or more memories as computer readable storage media storing instructions executable by the one or more processors for implementing various techniques and methods described herein. The photodetector and converting system may be implantable and coupled to or positioned near a medical device comprising the sensor system. In other aspects, the photodetector and converting system may be external and may include a wearable or handheld device. In various instances, the electrical signal output by the converting system is transmitted as radio waves to a radio receiver, optionally, a mobile phone. The detectable signal in some aspects is transmitted to a mobile phone, a wearable photodetector, or other computing device having one or more processors and one or more memories. Optionally, the wearable photodetector is a smart watch. In exemplary aspects, the detectable signal is detected or measured transcutaneously. See, e.g., FIG. 12.

The presently disclosed sensor systems, biomaterials, medical devices and solid supports may be made by any suitable methods known in the art. The art of chemical conjugation, gel formation, polymer crosslinking and the like are known techniques. Exemplary methods of making the presently disclosed sensor systems, biomaterials, and medical devices are described herein in the EXAMPLES section.

Methods of Use

The present disclosure further provides methods of determining a level of expression of a gene, an RNA, or a protein, or a combination thereof, in a subject. Optionally, a level of expression of a protein is determined. In exemplary aspects, the method comprises (i) implanting into the subject a presently disclosed biomaterial or medical device comprising a sensor system, optionally, a gel or a scaffold or other implant, at an implantation site within the subject, wherein the TBMs of the sensor system bind to the gene, RNA or protein for which the level of expression is being determined, and (ii) detecting or measuring the detectable signal produced by the sensor system to determine the level of expression of the target at the implantation site. In various aspects, the detectable signal is detected or measured transcutaneously.

In various instances, the detectable signal is continuously detected or measured over a time period. In various aspects, the time period comprises one or more time points before, during, and/or after treatment of the subject for a disease. In such embodiments, efficacy of the treatment may be monitored or tracked. In exemplary aspects, the time period comprises one or more time points before, during, and/or after diagnosis of the subject for a disease. In such embodiments, the disease progression, regression, and/or stage may be monitored. Optionally, the detectable signal is detected or measured hourly, daily, twice daily, three times daily, four times daily, every other hour, or weekly, bi weekly, or ever 3, 4, 5, or 6 days. In various instances, the detectable sign detected or measured monthly or bi monthly or quarterly or annually. In various aspects, the measured level of expression of the gene, RNA or protein is compared to a control level, wherein the measured expression level of the gene, RNA or protein, relative to the control level, is indicative of a disease status or efficacy of disease treatment.

In some instances, the method comprise measuring the expression level of at least two genes, RNA, or proteins in the subject by detecting or measuring at least two distinct detectable signals produced by the sensor system. In various aspects, the method comprises measuring the expression level of a plurality of genes, RNA, or proteins in the subject by detecting or measuring a plurality of distinct detectable signals produced by the sensor system. In various aspects, the method comprises measuring the level of expression of at least two proteins or a plurality of proteins.

The present disclosure also provides methods of detecting a disease in a subject. In exemplary embodiments, the method comprises determining a level of expression of a gene, an RNA, or a protein, or a combination thereof, in a subject in accordance with the presently disclosed methods of determining a level of expression of a gene, an RNA, or a protein, or a combination thereof, in a subject. Also provided are methods of monitoring progression, regression, or stage of a disease in a subject. In exemplary aspects, the method comprises determining a level of expression of a gene, an RNA, or a protein, or a combination thereof, in a subject in accordance with the presently disclosed methods of determining a level of expression of a gene, an RNA, or a protein, or a combination thereof, in a subject. In various aspects, the determining step occurs at a first time point and at a second time point, wherein the expression level measured at the first time point is compared to the expression level measured at the second time point, wherein the difference in the level of expression at the second time point relative to the level of expression at the first time point is indicative of progression, regression, or stage of the disease.

Methods of determining treatment for a subject with a disease are furthermore provided herein. In exemplary aspects, the method comprises monitoring progression, regression, or stage of the disease in the subject in accordance with the presently disclosed methods and determining the treatment based on the stage of the disease. Methods of determining efficacy of a treatment for a disease in a subject are also provided. In exemplary embodiments, the method comprises monitoring progression, regression, or stage of the disease in a subject in accordance with the presently disclosed methods, wherein the first time point occurs before treatment and the second time point occurs after treatment.

Methods of treating a disease in a subject are provided by the present disclosure. In exemplary aspects, the method comprises determining treatment for a subject with a disease, in accordance with the presently disclosed methods, and administering the treatment to the subject based on the outcome of the monitoring of the disease.

Further provided are methods of determining a level of expression of a gene, an RNA, or a protein, or a combination thereof, in a cell culture or tissue culture. In various aspects, the method comprises (i) contacting a presently disclosed solid support comprising a sensor system with a sample of a cell culture or tissue culture, wherein the TBMs of the sensor system bind to the gene, RNA or protein, and (ii) detecting or measuring the detectable signal produced by the sensor system to determine the level of expression of the target in the cell culture or tissue culture.

The following examples are given merely to illustrate the present invention and not in any way to limit its scope.

### EXAMPLES

### Example 1

This example describes an exemplary method of making a presently disclosed gel composition comprising a sensor system.

A gel composition comprising a sensor system capable of detecting or measuring expression of two targets, VEGF-A and S100a9, was made. The expression of VEGF-A and S100a9 are of interest to researchers, because S100a9 has been shown to play a role in cancer metastasis and inflammation and the role of VEGF-A in angiogenesis has been previously described. Since each of these proteins exists in vivo as a homodimer, each protein has two potential binding sites and therefore only one TBM is required for purposes of making a sensor system capable of detecting or measuring their expression. In this sensor system, the TBMs were peptides. The TBM peptide specific for S100a9 comprised the amino acid sequence CGMEWSLEKGYTIKGC(SEQ ID NO: 1) and the TBM peptide specific for VEGF-A comprised the amino acid sequence CGE{D-Phe}{D-Ala}{D-Tyr}{D-Leu}IDFNWEYPASKGC (SEQ ID NO: 2). Fluorophores (Cy5.5 and Cy7) were used to label the TBM peptide in batches. In a first batch, Cy5.5 was conjugated to the TBM peptide specific for S100a9 and in a second batch Cy7 was conjugated to the S100a9 TBM. In a third batch, Cy5.5 was conjugated to the TBM peptide specific for VEGF-A and in a second batch Cy7 was conjugated to the VEGF-A TBM. For each labeling, the peptide TBMs were first solubilized in DMSO before addition of an equal volume of PBS. Next, maleimide-functionalized fluorophores (Cy5.5 or Cy7) were added to the peptide TBMs and the mixture was incubated for 1 hour at room temperature. Fluorophore-labeled peptides were stored at −20 C until use.

Fluorophore-labeled peptides were next attached to 4-arm PEG. An aqueous solution comprising 20 kDa, 4-arm maleimide-functionalized PEG was incubated with Fluorophore-labeled peptides in a molar ratio of 1:1:1 (PEG:Cy5.5 labeled peptide:Cy7 labeled peptide) for 1 hour at room temperature. This solution was subsequently dialyzed for 24 hours with 4 changes of water to remove any impurities before aliquoting. The fluorophore-labeled TBM peptide-PEG conjugates were then freeze-dried and stored at −20° C. for later use.

Unlabeled PEG was doped with fluorophore-labeled TBM peptide-PEG conjugates and then crosslinked to form a gel. Briefly, an aqueous solution of PEG was prepared by dissolving unlabeled PEG in PBS and doping in 300 micrograms of fluorophore-labeled TBM peptide-PEG conjugates per mL. Gels were crosslinked by the addition of 3 mg of crosslinking peptide (GCYKNRGCYKNRCG(SEQ ID NO: 3)) per mL. The final concentration of total PEG in the crosslinked gels was 3% (w/v).

### Example 2

This example describes an exemplary method of making a presently disclosed polymeric microporous scaffold comprising a sensor system.

Fluorophore-labeled TBM peptide-PEG conjugates were made as described in Example 1. An aqueous solution was prepared by dissolving unlabeled PEG in PBS and doping in 300 micrograms of fluorophore-labeled TBM peptide-PEG conjugates per mL. Gels were crosslinked by the addition of 3 mg of crosslinking peptide (GCYKNRGCYKNRCG (SEQ ID NO: 3)) per mL of the prepared aqueous solution. Immediately after the addition of crosslinking peptide, but prior to gelation, the prepared aqueous solution comprising the crosslinking peptide was pipetted onto the surface of a microporous PCL scaffold (5 mm in diameter, 2 mm height). Gelation then occurred on the surface of the scaffold, anchoring the fluorophore-labeled TBM peptide-PEG conjugates in place.

In exemplary embodiments, immediately after the addition of crosslinking peptide, but prior to gelation, the prepared aqueous solution comprising the crosslinking peptide is pipetted into the pores of the surface of a microporous PCL scaffold via microinjection. The microinjection in some aspects occurs by hand or by a microinjecting robot. Gelation within the pores of the scaffold occurs, and the fluorophore-labeled TBM peptide-PEG conjugates.

### Example 3

This example describes an exemplary method of making a presently disclosed implant comprising a sensor system.

An implant, e.g., a hip replacement implant is coated with the presently disclosed sensor system. The system could be prepared as described and implant dipped into gel before gelation occurs to provide a thin coating. Another method is to functionalize an implant surface with modified PEG. This could be achieved with numerous click chemistries including EDC-NHS, thiol maleimide, azide-alkyne, etc.

Fluorophore-labeled TBM peptide-PEG conjugates are made as described in Example 1. An aqueous solution is prepared in a container large enough to fit the implant by dissolving unlabeled PEG in PBS and doping in 300 micrograms of fluorophore-labeled TBM peptide-PEG conjugates per mL. Immediately after adding 3 mg of crosslinking peptide (GCYKNRGCYKNRCG (SEQ ID NO: 3)) per mL of the prepared aqueous solution, the implant is dipped into the container comprising the prepared aqueous solution and the crosslinking peptide. Gelation then occurs on the surface of the implant, anchoring the fluorophore-labeled TBM peptide-PEG conjugates in place.

### Example 4

This example describes the binding capability of the fluorophore-labeled peptides and the capability of the FRET signal to represent target concentration.

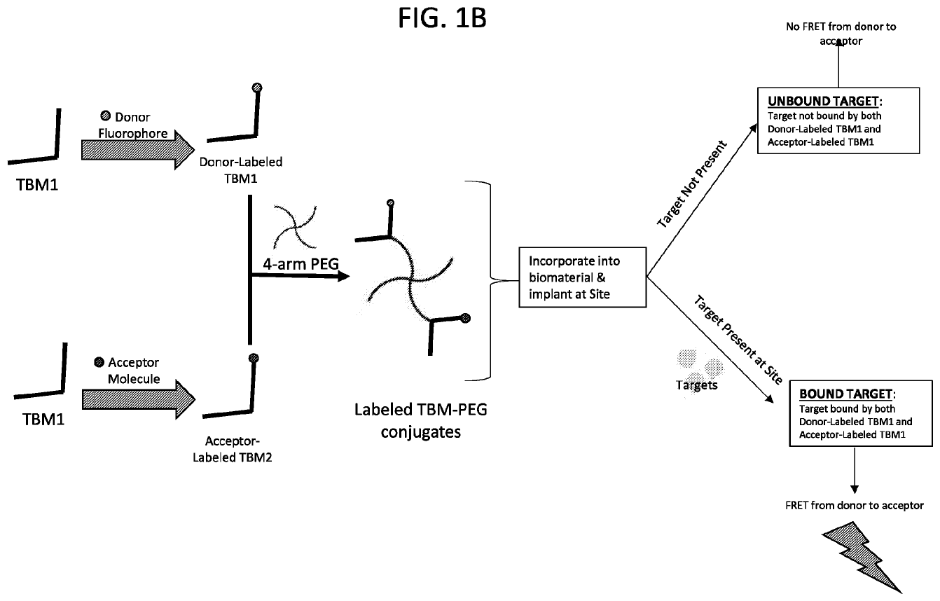

VEGF-A peptides labeled with Cy5.5 or Cy7 were made as essentially described in Example 1, and the ability of the labeled peptides to bind to the VEGF-A target was assayed in vitro. Briefly, 8 μg/mL of each TBM peptide (Cy5.5-labeled S100a9 TBM peptide and Cy7-labeled S100ap peptide) was added to a well of a 96-well plate containing PBS with 1% BSA. A solution comprising VEGF-A at varied concentrations (0-5000 ng/mL) was then added to the wells and the plates were incubated at room temperature for 1 hour. IVIS® imaging was carried out to measure emission of Cy7 upon excitation of Cy5.5 which was representative of the FRET from donor to acceptor. Intensity was determined for each well and the signal intensity was plotted as a function of VEGF-A concentration in FIG. 2A. As shown in this figure, the signal intensity increased with increasing concentration of VEGF-A.

This example demonstrates that the fluorophore-labeled VEGF-A peptides were functionally active in binding to the target and that the FRET signal represented concentration of the bound target.

### Example 5

This example describes a gel composition comprising a VEGF-A-specific sensor system and its ability to bind to its target (VEGF-A) and produce a FRET signal.

A gel composition comprising a sensor system comprising TBMs for VEGF-A were made as essentially described in Example 1. An aliquot of the gel composition was placed into wells of a multi-well plate and subsequently exposed to a solution comprising VEGF-A at varied concentrations (0 ng, 100 ng or 1000 ng). One row of wells on the multi-well plate contained gel compositions with a sensor system comprising VEGF-A peptides labeled with only the acceptor chromophore (Cy 5.5), and in another row were scaffolds with a sensor system comprising VEGF-A peptides labeled with only donor chromophores (Cy7). These two rows were controls. Each well of a third row comprised a gel composition with a sensor system comprising both VEGF-A peptides labeled with the acceptor chromophore and VEGF-A peptides labeled with donor fluorophores. The VEGF-A solutions were incubated with the gel compositions for 3 hours at room temp. IVIS® imaging was carried out to measure emission of Cy7 upon excitation of Cy5.5 which was representative of the FRET from donor to acceptor. The results are shown in FIG. 2B and the scale of radiance of epi-fluorescence is shown.

As shown in FIG. 2B, only the row comprising both the VEGF-A peptides labeled with donor fluorophore and VEGF-A peptides labeled with acceptor fluorophore produced a FRET signal, and this signal increased upon increasing concentration of VEGF-A.

### Example 6

This example describes a gel composition comprising a S100a9-specific sensor system and its ability to bind to its target (S100a9) and produce a FRET signal.

A gel composition comprising a sensor system comprising TBMs for S100a9 were made as essentially described in Example 1 and an aliquot was placed into a well of a multi-well plate. Culture media comprising S100a9 target was obtained by culturing cells from 4T1 tumor-bearing mice in culture media. The culture media comprising S100a9 was added to a well comprising the gel composition at (A) full strength (4T1 splenocyte conditioned media) or (B) diluted with control media (lacking S100a9) at a 1:1 ratio (1:1). Control media was added to a third well comprising a gel composition. The gel composition was incubated with the S100a9-containing media or control media for 3 hours at room temperature. IVIS® imaging was carried out to measure emission of Cy7 upon excitation of Cy5.5 which was representative of the FRET from donor to acceptor. The results are shown in FIG. 2C. As shown in this figure, the most intense FRET signal was obtained from the scaffold exposed to the full strength S100a9-containing media.

This example demonstrated that the scaffolds of the present disclosure are useful detecting and measuring target proteins in vitro.

### Example 7

This example describes implantation of a gel composition comprising a sensor system.

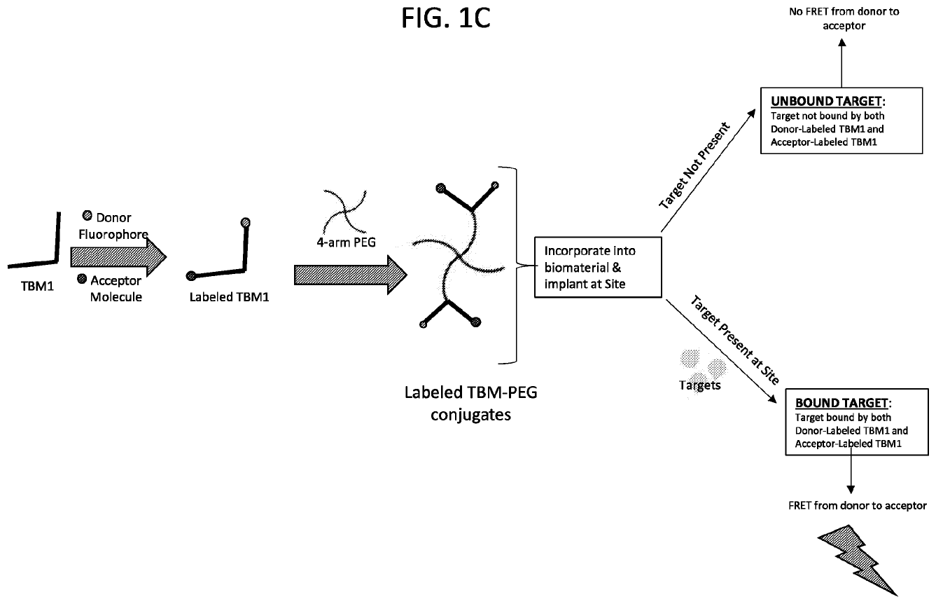

The gel compositions comprising a sensor system described in Example 5 were soaked in a solution comprising 1000 ng VEGF-A or 0 ng VEGF-A, as a control, for 24 hours at room temperature. The scaffolds were then subcutaneously implanted into a mouse dorsum via a small incision and stapled closed. Using an in vivo imaging system (IVIS®, Perkin Elmer), the signal was measured through the skin of the mouse. The IVIS® contains a fluorescence light source sufficient to excite the fluorophores through the skin and a photodetector sufficient to detect fluorescence. Each anesthetized mouse was placed in the chamber of the IVIS® system and imaged. Bandpass filters were used to select excitation and emission wavelengths for each fluorophore (Cy5.5, Cy7) or for the FRET system (excite Cy5.5 and emit Cy7). FIG. 3 shows the FRET signal as captured via IVIS imaging for mice implanted with gel compositions comprising a VEGF-A sensor system incubated with 0 ng or 1000 ng VEGF-A prior to implantation. As shown in this figure, the amount of signal increased with increasing VEGF-A concentration.

This example showed that protein concentration may be detected or measured in vivo and transcutaneously, supporting that the gel compositions of the present disclosure are useful for in vivo detection and measurement of a target protein.

### Example 8

This example describes implantation of a polymeric hydrogel comprising a sensor system which detects expression of two targets and further comprising a therapeutic agent.

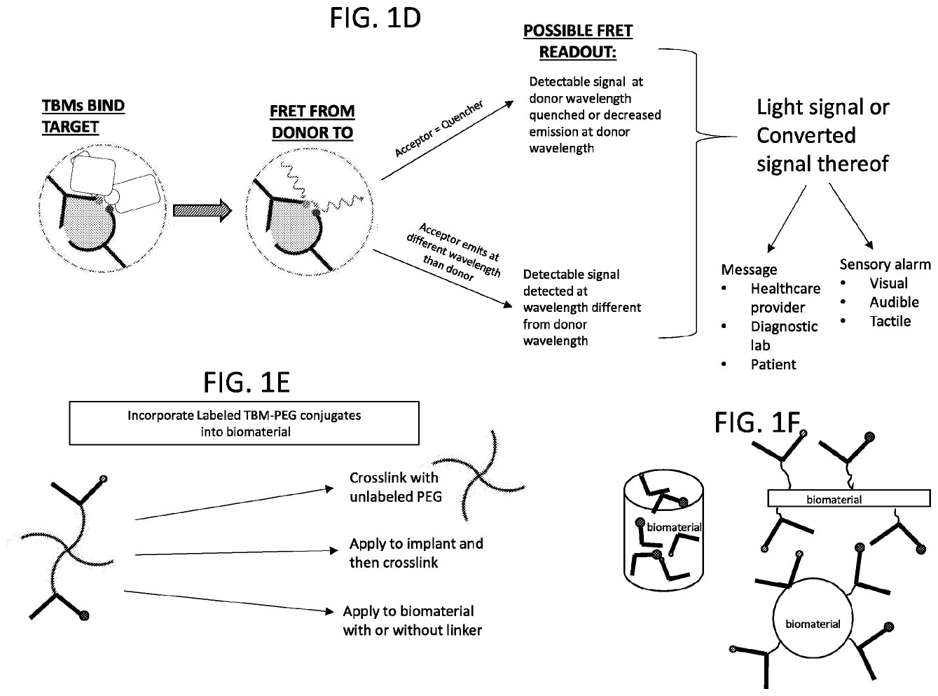

A hydrogel-based sensor system were made as essentially described in Example 2. Prior to gelation, an amount of dexamethasone (Dex), which reduces VEGF-A expression, or an amount of IFN-gamma, which increases VEGF-A expression, was doped into the aqueous solution comprising the fluorophore-labeled TBM peptide-PEG conjugates. Crosslinking peptide was added to the prepared aqueous solutions comprising conjugates alone, conjugates with Dex or conjugates with IFN-gamma. Three hydrogels were then subcutaneously implanted into a mouse at distinct sites: a control scaffold (Ctl, not doped with Dex or IFN-gamma) near the left forelimb, a scaffold doped with Dex near the mid-back, and a scaffold doped with IFN-gamma near the right forelimb. Transcutaneous images of the signals were taken on Day 0, Day 1, and Day 5 (post-implantation) via IVIS imaging, as essentially described in the previous example. The results are shown in FIG. 4. As shown in the top row of images of FIG. 4, over time, the FRET signal became stronger at the sites where IFN-gamma-doped scaffolds were implanted.

This example further supports that target protein concentration may be detected or measured in vivo and transcutaneously and further demonstrated that drugs incorporated into the biomaterial can change the amount of target being expressed.

### Example 9

This example describes implantation of a polymeric porous scaffold comprising a sensor system which detects expression of two targets and further comprising a therapeutic agent.

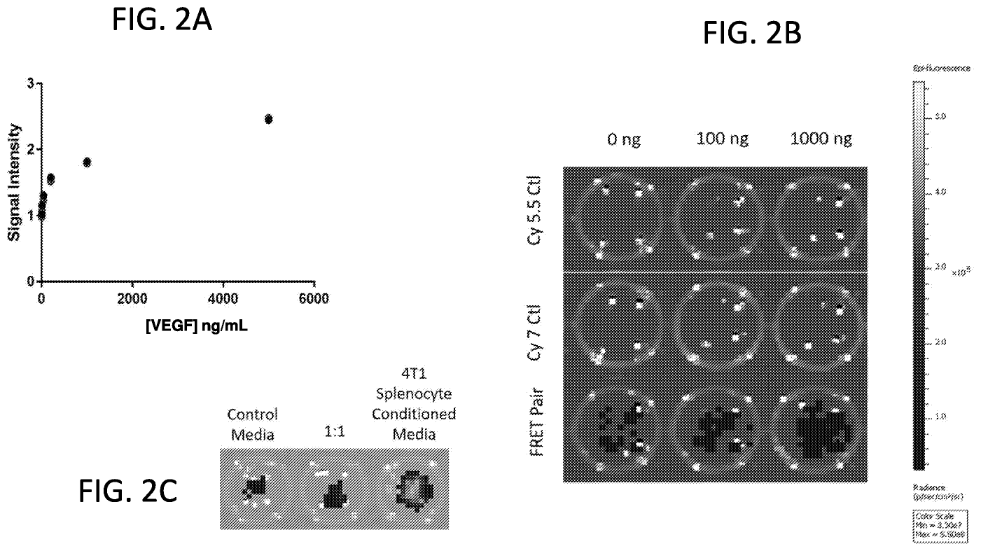

Scaffolds comprising a sensor system comprising TBMs specific for S100a9 were made as essentially described in Example 2. Breast cancer was induced in mice by orthotopic inoculation of 4T1 mammary carcinoma cells into the fourth right mammary fat pad. Twenty days post-tumor implantation, the scaffolds comprising a sensor system were implanted to monitor protein levels at the scaffold implantation site. Twenty-four hours post-scaffold implantation, mice were transcutaneously imaged via IVIS as essentially described above. As shown in FIG. 5A, tumor bearing (TB) mice (inside boxes) exhibited a higher FRET signal compared to tumor free mice (TF) (outside boxes). FIG. 5B provides a graph of the normalized intensity for the TB and TF mice. These results are consistent with our previous findings that tumor-bearing mice exhibit increased expression of S100a9.

This example supports that the presently disclosed scaffolds comprising a sensor system are useful for detecting, measuring and/or monitoring in vivo expression levels of target proteins in diseased and healthy animals.

### Example 10

This example describes an exemplary method of monitoring proteins expressed during wound healing.

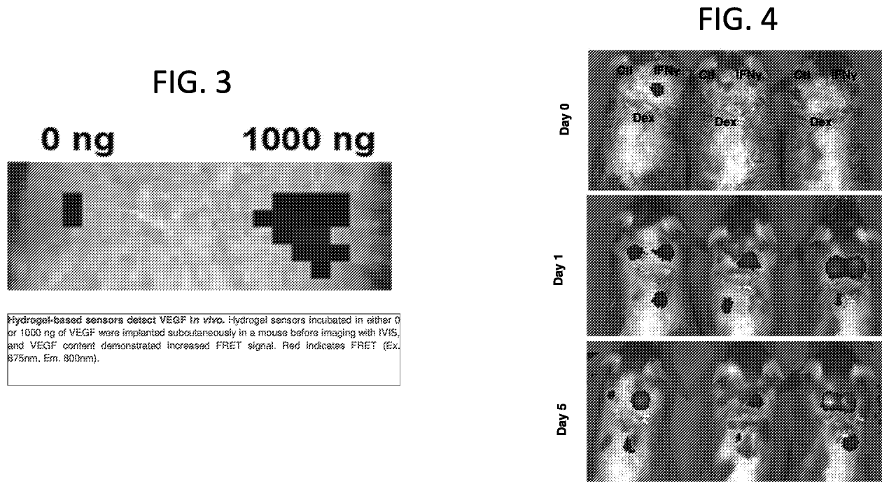

Gel compositions comprising TBMs specific for VEGF were made as essentially described in Example 1. The gel compositions were applied to full-thickness dermal wounds in mice and allowed to gel in situ before covering via a clear bandage (TegaDerm). The FRET signals were measured by IVIS imaging as essentially described in previous examples. The results are shown in FIG. 6.

As shown in FIG. 6, the intensity (correlating with VEGF expression) increased in the days immediately following wounding and then the intensity reduced. These trend of VEGF-A increasing and then decreasing during wound healing is consistent with prior studies.

This example supports that the gel compositions of the present disclosure can monitor dynamic VEGF expression during an injury/repair process in vivo.

### Example 11

This example describes an exemplary scaffold comprising a sensor system useful for detecting or measuring expression of genes of an autoimmune disease gene expression signature.

Prior research in our laboratory allowed for the identification of a gene expression signature (GES) representative of relapsing-remitting multiple sclerosis (RR-MS). The data supported that the following genes reliably change in the EAE mouse model, which is an animal model for RR-MS: Prtn3, Ela2, Ltf, Camp, Chi313, S100a8, S100a9, II8rb, Csf3, H2Q10, Cd55, Lefty, Ccl22, II12b, and Foxp3. Our prior studies also supported that the following 21 genes reliably change in scaffolds during EAE progression: Fn1, Lif, Bdkrb1, Tnfrsf11b, Cfb, Olr1, Clec7a, Cxcl5, Ptgs2, Cxcl3, II1f9, II1b, Trm1, Cxcl2, S100a9, Cxcl1, 116, Ereg, Vegfa, Cd163, Adrb2. A gel composition comprising a sensor system, optionally, placed atop a scaffold, capable of detecting and/or measuring a combination of the genes of the RR-MS GES, is made by using peptides, antibodies (or antigen-binding fragments thereof), aptamers or other antigen-binding proteins, linked to an acceptor or donor chromophore, e.g., Cy7, Cy5.5, wherein the chromophore-labeled peptides, antibodies (or antigen-binding fragments thereof), aptamers or other antigen-binding proteins collectively bind to several proteins expressed by the genes of the GES. For example, for Cxcl5, the Cxcl5 antibody, (Abcam, Cambridge, MA), is conjugated to a donor or acceptor chromophore and conjugated to a PEG. Other antibodies specific to the proteins expressed by genes of the above GES are commercially available from e.g., Abcam, among other vendors and manufacturers.

Once the scaffolds comprising the sensor system is made, it is used for in vitro detection and measurement of the target proteins (expressed by the genes of the GES) as essentially described in previous examples. Briefly, the scaffolds are exposed to solutions containing the target proteins and the FRET signals are imaged and quantified. The scaffolds are tested for in vivo detection and measurement as essentially described in previous examples.

FRET signals may be correlated with pre-determined expression levels of the proteins expressed by the genes of the GES. The pre-determined expression levels may be those ascertained by ELISA.

The genes of this gene expression signature that can be monitored in vivo, in real time in subjects with risk for the AID or subjects diagnosed with the AID using the presently disclosed scaffolds comprising a sensor system designed to detect the expression of a combination of the genes of the AID signature. In exemplary instances, the AID signature may be monitored in vivo in real time in subjects before, during and after treatment for the AID in order to determine the efficacy of the AID treatment.

To monitor disease progression, EAE mice are subcutaneously implanted with presently disclosed scaffolds comprising a sensor system. At Day −14, a polymer scaffold comprising polycaprolactone (PCL) comprising a sensor system comprising TBMs which bind to any or multiple of the above genes is implanted into the subcutaneous space of mice. In some studies, the procedure is carried out in experimental autoimmune encephalomyelitis (EAE) mice having a Swiss Jim Lambert (SJL) background. The EAE mouse model is a well-known animal model for relapsing-remitting multiple sclerosis (RR-MS). The foreign body response (FBR) to the implanted scaffolds is allowed to normalize in vivo for two weeks. In one study, disease is induced by immunization of mice with proteolipid protein peptide 139-151 (PLP) in complete Freund's adjuvant (CFA). The control group of mice in this study is immunized with an ovalbumin peptide (termed OVA). In another study, disease is induced with adoptively transferred autoreactive T-cells which recognize PLP. The control group of mice in this study is given autoreactive T-cells which recognize the OVA peptide. The mice are evaluated for symptoms of the disease including body condition and given a clinical score reflective of their coordination and level of paralysis which results from autoimmune-induced damage to neural tissue. Disease symptoms are expected to begin to show about 7 days after immunization of mice. It is expected that on Day 7 mice are pre-symptomatic (e.g., do not exhibit any signs or symptoms of disease) and thus have a clinical score of zero. On Day 9, mice are expected to exhibit symptoms and thus the clinical score on this day was about 3.5. In one study, as described below, mice are evaluated on Day 13, instead of Day 9.

The procedure described above is carried out with EAE mice. To induce disease in one group of EAE mice, a PLP peptide in complete Freund's adjuvant is injected into the mice. Healthy mice are used as a control. FRET signals from the scaffolds are measured daily. Protein expression data represented by FRET signal data are collected by IVIS and the FRET signals are assigned a score associated with probability of disease state.

To monitor efficacy of RR-MS treatment, FRET signals are measured before, during and after treatment of mice with standard-of-care RR-MS treatments: IFN-β, fingolimod, and an anti-VLA-4 antibody (non-humanized analogue of natalizumab: hereafter referred to simply as natalizumab).

### Example 12

This example describes an exemplary scaffold comprising a sensor system useful for detecting or measuring expression of genes of a diabetes gene expression signature.

To monitor diabetes progression, a scaffold comprising a sensor system capable of detecting and/or measuring the expression levels of the following 21 genes is made: Cd163, Ptgs2, Tnfrsf11b, Vegfa, Fn1, 116, Bdkrb1, S100a9, Cxcl1, Cxcl3, Cfb, Clec7a, II1b, II1f9, Cxcl5, Olr1, Lif, Cxcl2, Trem1, Ereg, Adrb2. After implanting the scaffolds in NOD mice, FRET signals generated at the scaffold implantation sites are monitored daily.

To monitor efficacy of treatment, FRET signals are monitored before, during and after treatment of mice with diabetes treatments including but not limited to an alpha-glucosidase inhibitor, biguanide, dopamine agonist, Dipeptidyl peptidase-4 inhibitor, GLP-1 receptor agonists, meglitinides, sodium-glucose transporter (SGLT) 2 inhibitors, sulfonylurea, thiazolidinediones, insulin, and the like.

### Example 13

This example describes an exemplary scaffold comprising a sensor system useful for detecting or measuring expression of genes of a metastatic disease gene expression signature.

To monitor metastatic disease status, a scaffold comprising a sensor system capable of detecting and/or measuring the expression levels of the following genes is made: S100 Calcium Binding Protein A8 (S100a8), S100 Calcium Binding Protein A9 (S100a9), Peptidoglycan Recognition Protein 1 (Pglyrp1), Lactotransferrin (Ltf), Cathelicidin Antimicrobial Peptide (Camp), Elastase 2 (Ela2), Chitinase (Chi313), Bone Morphogenetic Protein 15 (Bmp15), C-C Motif Chemokine Ligand 22 (Ccl22), C-C Motif Chemokine Receptor 7 (Ccr7). After implanting the scaffolds in mice, FRET signals generated at the scaffold implantation sites are monitored daily.

To monitor efficacy of treatment, FRET signals are monitored before, during and after treatment of mice with metastatic disease treatments.

### Example 14

This example describes the synthesis and characterization of a dual labeled TBM. The dual labeled TBM was a DNA aptamer comprising luciferase attached at one end of the aptamer and a quencher attached to the opposite end of the aptamer. The resulting dual labeled aptamer was studied for its ability to act as a sensor in a hydrogel. This example demonstrated a progression starting with the synthesis of the dual labeled TBM and its characterization in solution, moving to the attachment of the dual labeled TBM to a PEG macromer and the testing of the functionality of the dual labeled TBM attached to the PEG macromer, and then moving to the construction and testing of a hydrogel sensor comprising the dual labeled TBM. The results showed that each step of the progression was successful. Specifically, the dual labeled TBM specifically detected the exemplary target molecule (interferon-γ) in solution, when attached to a PEG macromer, and as part of a hydrogel sensor.

An interferon-γ (IFNg) binding DNA aptamer hairpin was synthesized to have the following sequence: Biotin-GG GGT TGG TTG TGT TGG GTG TTG TGT CCA ACC CC-Azide (SEQ ID NO: 4). Next, recombinant firefly luciferase was incubated with an amine-reactive NHS ester form of dibenzocyclooctyne (DBCO) in a 1:8 molar ratio (luciferase:DBCO) in 100 mM sodium bicarbonate. This reaction tags amines on the luciferase with a cycloalkyne for strain promoted alkyne azide cycloaddition. Modified luciferase was purified using Amicon spin columns.

In a separate reaction recombinant streptavidin was modified with an amine-reactive quencher (IRDye® QC-1 from LICOR) (1:8 molar ratio in 100 mM sodium bicarbonate). Modified streptavidin was purified using Amicon spin columns.

To make quencher and luciferase modified aptamer, DNA aptamer was first reacted (room temperature) with DBCO-modified luciferase for 15 minutes in a 1:1 molar ratio. Then it was reacted for 30 minutes with quencher-labeled streptavidin (1:1). This “modified aptamer” was used to conduct all studies in this Example. See FIG. 7A.

To test the modified aptamer in solution, a plate was prepared with wells containing 100 uL total volume. In this volume was contained 3.5 nM modified aptamer and various concentrations of IFNg or 500 nM DNA complement. The buffer for the reaction was T4 ligase buffer from New England Biolabs, because it contains all necessary components for luciferase reaction. Plates were incubated for 30 minutes at room temperature before addition of luciferin to a final concentration of 630 nM. Plates were then immediately imaged using an In Vivo Imaging System to measure luminescent activity. See FIG. 7B.

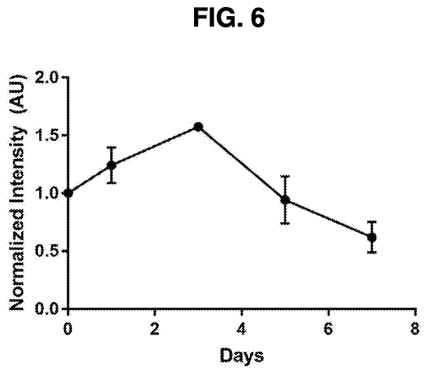

To make modified aptamer conjugated PEG macromers, a 4-arm, 40 kDA PEG macromer with maleimide functional groups was reacted with a 1 kDa Biotin-PEG-Thiol (1:1 molar ratio) for 30 minutes at room temperature. This macromer was then reacted with the modified aptamer for 30 minutes at room temperature. This resulted in a PEG macromer functionalized with biotin bound to streptavidin that is bound to the biotin functionalized DNA aptamer (each streptavidin molecule can bind up to four biotins). Then tests of aptamer functionality were carried out as described above. See FIG. 8.

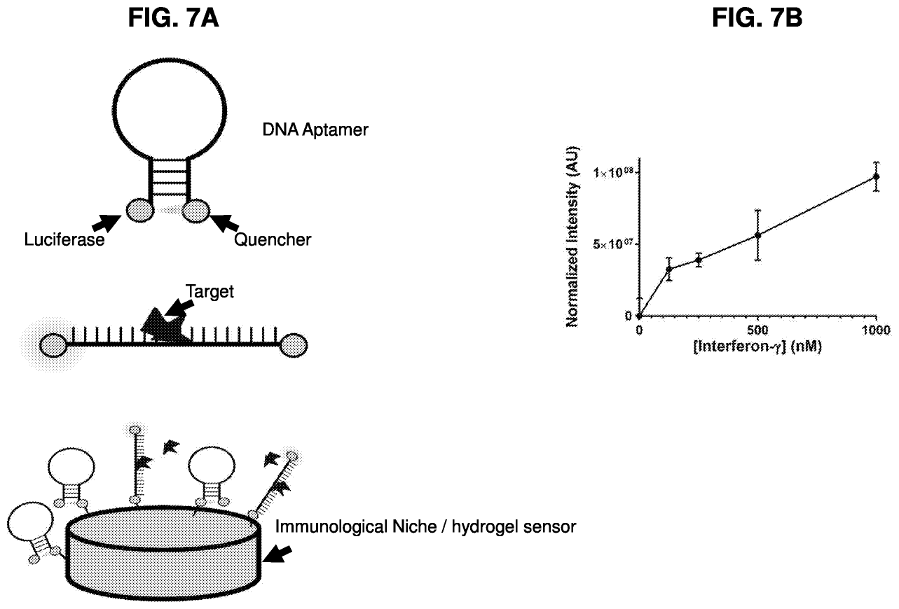

To make modified aptamer conjugated hydrogel sensors a 10% PEG solution was made (with a ratio of 1:25 modified:unmodified PEG macromer). This solution was then mixed 1:1 with 3.33 mM crosslinking peptide (dYNKD—sequence: GC{d-TYR}K{d-ASN}RGC{d-TYR}K{d-ASN}RCG) (SEQ ID NO: 5) to form a hydrogel. d-ASN and d-TYR are the d-enantiomers of asparagine and tyrosine, respectively. They are non-natural amino acids, used to slow degradation. Each hydrogel also contained either 1 uM IFNg, 0 uM IFNg, or 500 nM DNA complement. After allowing hydrogels to gel for 30 minutes at 37 C, 100 uL of T4 ligase buffer and incubated for 1 hour at 37 C. Then luciferin was added and gels were imaged as described above. See FIG. 9. As is clear from FIGS. 7-9, the modified aptamer readily detected IFNg when attached to a PEG hydrogel sensor by emission of light. These sensors can be readily deployed for measurement of IFNg in vivo. Additionally, the aptamer sequence may be modified to be specific to any number of other targets (e.g., proteins).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A biomaterial comprising a sensor system comprising a donor linked to a target binding moiety (TBM) and an acceptor molecule linked to a TBM, wherein, when the TBM linked to the donor and the TBM linked to the acceptor molecule binds to a target, a resonance energy transfer (RET) from the donor to the acceptor molecule (i) occurs or (ii) decreases or stops occurring and a detectable signal is produced.

2. The biomaterial of claim 1, wherein the RET is Forster resonance energy transfer (FRET), bioluminescent resonance energy transfer (BRET), chemiluminescent resonance energy transfer (CRET), or a combination thereof.

3. The biomaterial of claim 1 or claim 2, wherein the TBM linked to the donor is also linked to the acceptor molecule.

4. The biomaterial of claim 1 or claim 2, wherein the sensor system comprises a first TBM linked to the donor and a second TBM linked to the acceptor molecule.

5. The biomaterial of claim 4, wherein the first TBM and the second TBM are structurally the same.

6. The biomaterial of claim 4, wherein the first TBM and the second TBM are structurally different.

7. The biomaterial of claim 6, wherein the first TBM binds to a binding site on the target distinct from the binding site of the second TBM.

8. The biomaterial of any one of claims 1-7, wherein the donor is a donor fluorophore, a donor luminogenic protein, a donor chemiluminescent compound, or a combination thereof.

9. The biomaterial of claim 8, wherein the acceptor molecule comprises an acceptor fluorophore which emits fluorescence at a wavelength different from the wavelength at which the donor fluorophore emits fluorescence, and the detectable signal is fluorescence emitted at the acceptor fluorophore wavelength.

10. The biomaterial of claim 8, wherein each of the acceptor fluorophore and donor fluorophore emits fluorescence at a wavelength within a range of about 550 nm to about 900 nm, optionally, about 650 nm to about 870 nm.

11. The biomaterial of claim 8, wherein the acceptor molecule comprises a quencher moiety which quenches the fluorescence emitted by the donor fluorophore, and the detectable signal is a quenched fluorescence at the wavelength at which the donor fluorophore emits fluorescence.

12. The biomaterial of claim 8, wherein the acceptor molecule comprises a quencher moiety which quenches the luminescence emitted by the donor luminogenic protein, and the detectable signal is a luminescence at the wavelength at which the donor luminogenic protein emits luminescence.

13. The biomaterial of claim 11, wherein the quencher comprises a metal ion.

14. The biomaterial of claim 11, wherein the quencher is a quencher fluorophore.

15. The biomaterial of claim 12, wherein the quencher comprises an inhibitor of enzyme activity.

16. The biomaterial of any one of the preceding claims, wherein the target is a protein.

17. The biomaterial of any one of the preceding claims, wherein the TBM is a peptide, protein, or aptamer.

18. The biomaterial of claim 13, wherein the peptide is less than about 100 amino acids in length, optionally, less than about 75 amino acids in length.

19. The biomaterial of claim 13, wherein the protein comprises an antibody or an antigen-binding fragment thereof, optionally, a Fab fragment.

20. The biomaterial of claim 13, wherein the protein is an antibody protein product, optionally, a nanobody, a camelid, or an scFv.

21. The biomaterial of any one of the preceding claims, wherein all of the TBMs of the sensor system bind to the same target.

22. The biomaterial of any one of claims 1 to 20, wherein a subset of the TBMs of the sensor system binds to a first target and at least one other subset of the TBMs of the sensor system binds to a second target, wherein a first detectable signal is produced when the first target is bound and a second detectable signal is produced when the second target is bound, wherein the first detectable signal is distinct from the second detectable signal.

23. The biomaterial of claim 22, wherein the TBMs of the sensor system collectively bind to three or more targets, wherein a distinct detectable signal is produced for each target.

24. The biomaterial of claim 23, wherein the TBMs of the sensor system collectively bind to 5 to 10 targets, wherein, for each target, a distinct detectable signal is produced upon binding of the TBMs to its target.

25. The biomaterial of claim 24, wherein the TBMs of the sensor system collectively bind to more than 10, more than 25, or more than 50 targets, wherein, for each target, a distinct detectable signal is produced upon binding of the TBMs to its target.

26. The biomaterial of claim 25, wherein the targets are gene products of genes of a gene expression signature (GES) indicative or predictive of a disease or a medical condition, or risk therefor, optionally, wherein the gene products are proteins.

27. The biomaterial of claim 26, wherein the disease or medical condition is diabetes, inflammation, multiple sclerosis (MS), transplant rejection, or cancer.

28. The biomaterial of claim 27, wherein the target are gene products of genes of a GES for metastatic cancer.

29. The biomaterial of claim 27, wherein the target are gene products of genes of a GES for RR-MS.

30. The biomaterial of any one of the preceding claims, wherein the detectable signal is detectable by an optical camera.

31. The biomaterial of any one of claims 2-30, further comprising a converting system which converts the FRET from the donor fluorophore to the acceptor molecule into an electrical signal.

32. The biomaterial of claim 31, wherein the converting system comprises a photodetector.

33. The biomaterial of claim 31 or 32, wherein the electrical signal is transmitted as radio waves to a radio receiver, optionally, a mobile phone.

34. The biomaterial of any one of the preceding claims, wherein the detectable signal is detected or measured transcutaneously.

35. The biomaterial of any one of the preceding claims, wherein the detectable signal is transmitted to a mobile phone, a wearable photodetector, or a computer.

36. The biomaterial of claim 35, wherein the wearable photodetector is a smart watch.

37. The biomaterial of any one of the preceding claims, wherein the sensor system is covalently attached to the biomaterial.

38. The biomaterial of any one of the preceding claims, wherein the sensor system is attached to the biomaterial via non covalent bonds.

39. The biomaterial of any one of the preceding claims, wherein the sensor system is encapsulated or housed by the biomaterial.

40. The biomaterial of any one of the preceding claims, wherein the sensor system is impregnated, saturated or infused throughout the biomaterial.

41. The biomaterial of any one of the preceding claims, wherein the biomaterial is porous and the sensor system is located in the pores of the biomaterial.

42. The biomaterial of any one of the preceding claims, comprising a polymer, optionally, a poly(ethylene glycol) (PEG).

43. The biomaterial of claim 42, wherein the PEG is 3-arm PEG, 4-arm PEG, or 8-arm PEG.

44. The biomaterial of claim 42 or 43, wherein at least one arm of the PEG is linked to a TBM linked to an acceptor molecule and/or a donor fluorophore.

45. The biomaterial of claim 44, wherein two or more arms of the PEG are linked to a TBM linked to an acceptor molecule and/or a donor fluorophore.

46. The biomaterial of any one of claims 42-45, wherein at least one arm of the PEG is linked to an arm of another PEG.

47. The biomaterial of any one of the preceding claims, comprising a crosslinked polymer.

48. The biomaterial of any one of the preceding claims, wherein the biomaterial is a gel optionally a hydrogel.

49. The biomaterial of any one of the preceding claims, further comprising a therapeutic agent, optionally, wherein the therapeutic agent is released from the biomaterial.

50. A medical device comprising a biomaterial of any one of the preceding claims.

51. The medical device of claim 50, which is an implant.

52. The medical device of claim 51, which is a polymeric scaffold.

53. The medical device of any one of claims 50-52, comprising a metal, plastic, glass, fabric, or silicon.

54. The medical device of any one of claims 50 to 53, comprising a material comprising a plurality of micropores.

55. The medical device of any one of claims 50-54, wherein the biomaterial, optionally, a gel, is adhered to the surface of the medical device.

56. The medical device of 55, wherein the biomaterial, optionally, a gel, is present in the pores of the medical device.

57. The medical device of claim 56, wherein the biomaterial is present only in the pores of the medical device.

58. The medical device of any one of claims 50 to 57, wherein the TBMs are located at a unique location of the medical device, wherein detection of the detectable signal at the unique location enables identification of the target bound by the TBMs.

59. A medical device comprising a sensor system comprising a donor linked to a target binding moiety (TBM) and an acceptor molecule linked to a TBM, wherein, when the TBM linked to the donor and the TBM linked to the acceptor molecule binds to a target, a resonance energy transfer (RET) from the donor to the acceptor molecule (i) occurs or (ii) decreases or stops occurring and a detectable signal is produced.

60. The medical device of claim 59, wherein the RET is Forster resonance energy transfer (FRET), bioluminescent resonance energy transfer (BRET), chemiluminescent resonance energy transfer (CRET), or a combination thereof.

61. The medical device of claim 59 or claim 60, wherein the donor is a donor fluorophore, a donor luminogenic protein, a donor chemiluminescent compound, or a combination thereof.

62. The medical device of any one of claims 59-61 which is a polymeric medical device.

63. The medical device of any one of claims 59-61 which is a naturally-derived medical device.

64. The medical device of any one of claims 59 to 63 which is a wound dressing.

65. A solid support attached to a sensor system comprising a donor linked to a target binding moiety (TBM) and an acceptor molecule linked to a TBM, wherein, when the TBM linked to the donor and the TBM linked to the acceptor molecule binds to a target, a resonance energy transfer (RET) from the donor to the acceptor molecule occurs and a detectable signal is produced, wherein the sensor system is in contact with a sample of a cell culture or a sample obtained from a subject.

66. The solid support of claim 65, wherein the RET is Forster resonance energy transfer (FRET), bioluminescent resonance energy transfer (BRET), chemiluminescent resonance energy transfer (CRET), or a combination thereof.

67. The solid support of claim 65 or claim 66, wherein the donor is a donor fluorophore, a donor luminogenic protein, a donor chemiluminescent compound, or a combination thereof.

68. The solid support of any one of claims 65-67, wherein the implant is made of a metal, plastic, glass, fabric, or silicon.

69. The solid support of claim 68, which is tissue culture plasticware.

70. The solid support of any one of claims 65-69, wherein the sensor system accords with any of the sensor systems described herein.

71. The solid support of any one of claims 65-70, which is functionalized with a moiety and the sensor system is covalently attached to the solid support via the moiety.

72. A method of determining a level of expression of a gene, an RNA, or a protein, or a combination thereof, in a subject, comprising (i) implanting into the subject a biomaterial or medical device of any one of claims 1-64, at an implantation site, wherein the TBMs of the sensor system bind to the gene, RNA or protein, and (ii) detecting or measuring the detectable signal produced by the sensor system to determine the level of expression of the target at the implantation site.

73. The method of claim 72, wherein the detectable signal is detected or measured transcutaneously.

74. The method of claim 72 or 73, wherein the detectable signal is continuously detected or measured over a time period.

75. The method of claim 74, wherein the time period comprises one or more time points before, during, and/or after treatment of the subject for a disease.

76. The method of claim 74, wherein the time period comprises one or more time points before, during, and/or after diagnosis of the subject for a disease.

77. The method of any one of claims 72-76, wherein the measured level of expression of the gene, RNA or protein is compared to a control level, wherein the measured expression level of the gene, RNA or protein, relative to the control level, is indicative of a disease status or efficacy of disease treatment.

78. The method of any one of claims 72-77, comprising measuring the expression level of at least two genes, RNA, or proteins in the subject by detecting or measuring at least two distinct detectable signals produced by the sensor system.

79. The method of claim 78, comprising measuring the expression level of a plurality of genes, RNA, or proteins in the subject by detecting or measuring a plurality of distinct detectable signals produced by the sensor system.

80. A method of detecting a disease in a subject, comprising determining a level of expression of a gene, an RNA, or a protein, or a combination thereof, in a subject according to any one of claims 72-79.

81. A method of monitoring progression, regression, or stage of a disease in a subject, comprising determining a level of expression of a gene, an RNA, or a protein, or a combination thereof, in a subject according to any one of claims 72-79 at a first time point and at a second time point, wherein the expression level measured at the first time point is compared to the expression level measured at the second time point, wherein the difference in the level of expression at the second time point relative to the level of expression at the first time point is indicative of progression, regression, or stage of the disease.

82. A method of determining treatment for a subject with a disease, comprising monitoring progression, regression, or stage of the disease in the subject in accordance with claim 81 and determining the treatment based on the stage of the disease.

83. A method of determining efficacy of a treatment for a disease in a subject, comprising monitoring progression, regression, or stage of the disease in a subject in accordance with claim 81, wherein the first time point occurs before treatment and the second time point occurs after treatment.

84. A method of treating a disease in a subject, comprising determining treatment for a subject with a disease, according to claim 82, and administering the treatment to the subject based on the outcome of the monitoring of the disease.

85. A method of determining a level of expression of a gene, an RNA, or a protein, or a combination thereof, in a cell culture or tissue culture, comprising (i) placing a solid support of any one of claims 65-71, in a cell culture or tissue culture, wherein the TBMs of the sensor system bind to the gene, RNA or protein, and (ii) detecting or measuring the detectable signal produced by the sensor system to determine the level of expression of the target in the cell culture or tissue culture.

86. The method of any one of claims 72-85, wherein a level of expression of a protein in a subject is determined.

87. The method of claim 86, wherein the expression levels of two or more proteins in a subject are determined, optionally, wherein the expression levels of a plurality of proteins in a subject are determined.


SEQUENCE LISTING (5 sequences)

SEQ ID NO: 1 - protein, 16 aa, Artificial Sequence
  Synthetic
CGMEWSLEKGYTIKGC

SEQ ID NO: 2 - protein, 20 aa, Artificial Sequence
  Synthetic
  MISC_FEATURE (4)..(4): D-Phe
  MISC_FEATURE (5)..(5): D-Ala
  MISC_FEATURE (6)..(6): D-Tyr
  MISC_FEATURE (7)..(7): D-Leu
CGEXXXXIDFNWEYPASKGC

SEQ ID NO: 3 - protein, 14 aa, Artificial Sequence
  Synthetic
GCYKNRGCYKNRCG

SEQ ID NO: 4 - DNA, 34 bp, Artificial Sequence
  Synthetic
  misc_feature (1)..(1): Biotin
  misc_feature (34)..(34): Azide
ggggttggttgtgttgggtgttgtgtccaacccc

SEQ ID NO: 5 - protein, 14 aa, Artificial Sequence
  Synthetic
  MISC_FEATURE (3)..(3): d-TYR
  MISC_FEATURE (5)..(5): d-ASN
  MISC_FEATURE (9)..(9): d-TYR
  MISC_FEATURE (11)..(11): d-ASN
GCXKXRGCXKXRCG